(19) United States
(12) Patent Application Publication
(10) Pub. No.: US 2023/0207109 A1
(43) Pub. Date: Jun. 29, 2023

(54) MEDICAL SUPPORT DEVICE, DISPLAY DEVICE, MEDICAL SUPPORT METHOD, AND PROGRAM

(71) Applicant: FUJIFILM Corporation, Tokyo (JP)

(72) Inventors: Sumire Kuroda, Tokyo (JP); Tsuyoshi Hirakawa, Tokyo (JP); Hiroshi Hiramatsu, Tokyo (JP); Jun Aizu, Tokyo (JP)

(73) Assignee: FUJIFILM Corporation, Tokyo (JP)

(21) Appl. No.: 18/068,488

(22) Filed: Dec. 19, 2022

(30) Foreign Application Priority Data
Dec. 24, 2021 (JP) 2021-211561

(51) Int. Cl. G16H 40/20; G16H 10/60; G06T 11/20

(52) U.S. Cl. CPC G16H 40/20; G16H 10/60; G06T 11/206; G06T 2210/41

(57) ABSTRACT

A medical support device includes at least one processor, in which the processor acquires hospitalization period medical information including a hospitalization period date, which is a date corresponding to a hospitalization period, and a plurality of first items associated with the hospitalization period date, and generates a display screen including a first display region defined by a first axis indicating the hospitalization period date and a second axis indicating the plurality of first items and intersecting with the first axis, in which the hospitalization period medical information is disposed in a matrix in the first display region.

### CROSS-REFERENCE TO RELATED APPLICATION

This application claims priority from Japanese Patent Application No. 2021-211561, filed Dec. 24, 2021, the disclosure of which is incorporated herein by reference in its entirety.

### BACKGROUND

### Technical Field

The technology of the present disclosure relates to a medical support device, a display device, a medical support method, and a non-transitory storage medium storing a program.

### Related Art

JP2018-010330A discloses a time-series data display control device comprising a screen data generation unit that generates a time-series data display screen on which a plurality of time-series data are arranged along a data arrangement axis orthogonal to a time axis and displayed, and a screen display controller that switches between a plurality of display modes including a graph display mode in which the time-series data is displayed in a form of a graph in which a position or a length of an input point of data fluctuates in a data arrangement axis direction in accordance with a value of the input data, and a dot display mode in which the time-series data is displayed in a form of a plurality of dots in which a plurality of input points of the data are arranged on a straight line parallel to the time axis.

JP2017-117265A discloses a medical care support device that provides a plurality of examination data in which an examination item, an examination value, and examination date and time of a medical examination performed on a patient are stored in association with each other, and a plurality of dose data in which a type, an administered dose, and an administration period of a drug administered to the patient are stored in association with each other, the device comprising an acquisition unit that acquires a related-data set including the dose data of at least one drug and the examination data of at least one examination item affected by the administration of the drug, and a screen output controller that controls output of a display screen including a plurality of related-data set display fields in which the administered dose and the examination value for the related-data set are arranged at adjacent positions and displayed in time series.

JP2017-027266A discloses an information analysis support device comprising a screen generation unit that generates an information display screen including a first display region in which a table in which two types of attributes for classifying information are assigned to an axis in a row direction or an axis in a column direction, respectively, is displayed and a second display region in which the information is displayed, and a screen output controller that controls output of the information display screen, in which the screen generation unit displays the information in the second display region in three different display formats in accordance with selection methods of a first selection method in which the attribute assigned to the axis of the row direction is selected, a second selection method in which the attribute assigned to the axis of the column direction is selected, and a third selection method in which a cell, which is an intersection of the two types of attributes, is selected.

JP2019-040367A discloses a medical information processing system comprising a storage unit that stores image data indicating medical information and non-image data, which is data other than the image data among data indicating the medical information, in association with a time, an acquisition unit that receives an operation with respect to the non-image data displayed in time series and acquires a time range corresponding to the operation, an extraction unit that extracts the image data in accordance with the time range from the storage unit, and a display controller that displays the image data extracted by the extraction unit.

### SUMMARY

One embodiment according to the technology of the present disclosure provides a medical support device, a display device, a medical support method, and a non-transitory storage medium storing a program which facilitate grasping hospitalization period medical information of a patient as compared with a case in which all medical information from the past to the present is displayed.

A first aspect according to the technology of the present disclosure relates to a medical support device comprising at least one processor, in which the processor acquires hospitalization period medical information including a hospitalization period date, which is a date corresponding to a hospitalization period, and a plurality of first items associated with the hospitalization period date, and generates a display screen including a first display region defined by a first axis indicating the hospitalization period date and a second axis indicating the plurality of first items and intersecting with the first axis, in which the hospitalization period medical information is disposed in a matrix in the first display region.

A second aspect according to the technology of the present disclosure relates to a medical support method comprising acquiring hospitalization period medical information including a hospitalization period date, which is a date corresponding to a hospitalization period, and a plurality of first items associated with the hospitalization period date, and generating a display screen including a first display region defined by a first axis indicating the hospitalization period date and a second axis indicating the plurality of first items and intersecting with the first axis, in which the hospitalization period medical information is disposed in a matrix in the first display region.

A third aspect according to the technology of the present disclosure relates to a non-transitory storage medium storing a program causing a computer to execute a process comprising acquiring hospitalization period medical information including a hospitalization period date, which is a date corresponding to a hospitalization period, and a plurality of first items associated with the hospitalization period date, and generating a display screen including a first display region defined by a first axis indicating the hospitalization period date and a second axis indicating the plurality of first items and intersecting with the first axis, in which the hospitalization period medical information is disposed in a matrix in the first display region.

### BRIEF DESCRIPTION OF THE DRAWINGS

FIG. 1 is a block diagram showing an example of a configuration of a medical support system.

FIG. 2 is a block diagram showing an example of a hardware configuration of an electric system of a medical support server and a client terminal.

FIG. 3 is a block diagram showing an example of a hardware configuration of an electric system of the medical support server, an electronic medical record server, an image management server, and a document management server.

FIG. 4 is a conceptual diagram showing an example of processing contents of a display controller, a generation unit, and a first transmission/reception unit of the client terminal in a case in which a screen creation condition or the like is received by the client terminal.

FIG. 5 is a conceptual diagram showing an example of processing contents of a third transmission/reception unit and a search unit of the electronic medical record server.

FIG. 6 is a conceptual diagram showing an example of processing contents of an acquisition unit and a second transmission/reception unit of the medical support server.

FIG. 7 is a conceptual diagram showing an example of processing contents of a fourth transmission/reception unit and a search unit of the image management server.

FIG. 8 is a conceptual diagram showing an example of processing contents of a fifth transmission/reception unit and a search unit of the document management server.

FIG. 9 is a conceptual diagram showing an example of processing contents of the acquisition unit, a screen generation unit, and the second transmission/reception unit of the medical support server, and processing contents of the first transmission/reception unit and the display controller of the client terminal.

FIG. 10 is a screen view showing an example of a display screen.

FIG. 11 is a screen view showing an example of the display screen.

FIG. 12 is a flowchart showing an example of a flow of screen display processing.

FIG. 13 is a flowchart showing an example of a flow of display screen generation processing.

FIG. 14 is a flowchart showing an example of a flow of electronic medical record management processing.

FIG. 15 is a flowchart showing an example of a flow of image management processing.

FIG. 16 is a flowchart showing an example of a flow of document management processing.

FIG. 17 is a screen view showing an example of an aspect in which medical information is displayed in a date order on the display screen.

FIG. 18 is a screen view showing an example of an aspect in which the medical information is displayed in a selection order on the display screen.

FIG. 19 is a screen view showing an example of an aspect in which a row is selected in a first display region.

FIG. 20 is a conceptual diagram showing an example of processing contents of the fourth transmission/reception unit and the search unit of the image management server.

FIG. 21 is a conceptual diagram showing an example of processing contents of the fifth transmission/reception unit and the search unit of the document management server.

FIG. 22 is a conceptual diagram showing an example of processing contents of the acquisition unit, the screen generation unit, and the second transmission/reception unit of the medical support server, and processing contents of the first transmission/reception unit and the display controller of the client terminal.

FIG. 23 is a screen view showing an example of an aspect in which a pre-hospitalization period is switched between display and non-display.

FIG. 24 is a conceptual diagram showing an example of processing contents of the acquisition unit, the screen generation unit, and the second transmission/reception unit of the medical support server, and processing contents of the first transmission/reception unit and the display controller of the client terminal.

FIG. 25 is a screen view showing an example of an aspect in which a post-hospital discharge period is switched between display and non-display.

FIG. 26 is a conceptual diagram showing an example of processing contents of the acquisition unit, the screen generation unit, and the second transmission/reception unit of the medical support server, and processing contents of the first transmission/reception unit and the display controller of the client terminal.

FIG. 27 is a screen view showing an example of an aspect in which a width of the first display region is adjusted in accordance with the hospitalization period.

FIG. 28 is a screen view showing an example of an aspect in which an upper limit value is set in the width of the first display region.

FIG. 29 is a conceptual diagram showing an example of processing contents of the acquisition unit, the screen generation unit, and the second transmission/reception unit of the medical support server, and processing contents of the first transmission/reception unit and the display controller of the client terminal.

FIG. 30 is a screen view showing an example of an aspect in which the first display region is switched to a last hospitalization period or a hospitalization period before the last.

FIG. 31 is a screen view showing an example of an aspect in which an icon is switched in accordance with a state of the medical information.

FIG. 32 is a screen view showing an example of an aspect in which the icon indicates the number of pieces of the medical information.

FIG. 33 is a screen view showing an example of an aspect in which the icon indicates the number of pieces of the medical information.

FIG. 34 is a screen view showing an example of an aspect in which the row is designated in a case in which the icon indicates the number of pieces of medical information.

FIG. 35 is a block diagram showing another example of the configuration of the medical support system.

### DETAILED DESCRIPTION

An example of an embodiment of a medical support device, a display device, a medical support method, and a program according to the technology of the present disclosure will be described with reference to the accompanying drawings.

### First Embodiment

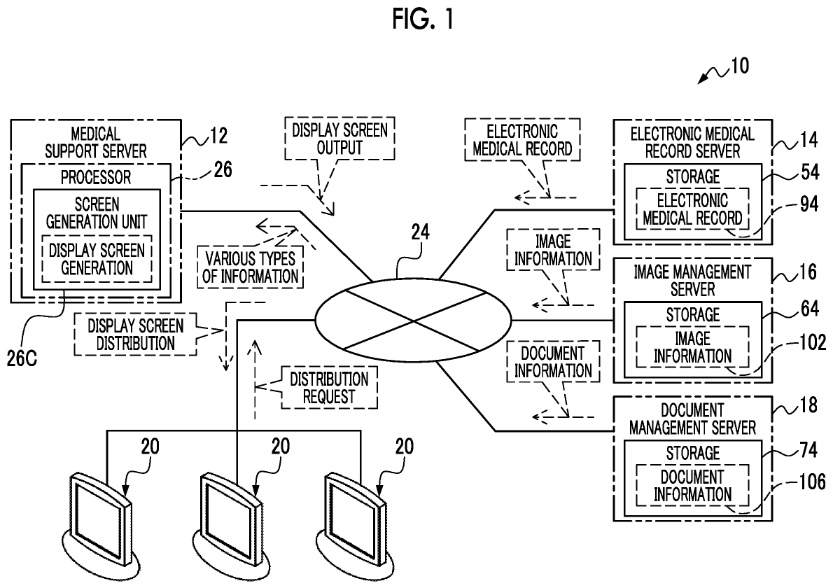

As shown in FIG. 1 as an example, a medical support system 10 comprises a medical support server 12, an electronic medical record server 14, an image management server 16, a document management server 18, and a client terminal 20. The medical support system 10 is a system used for at least one facility (for example, a hospital) that handles a plurality of pieces of medical information.

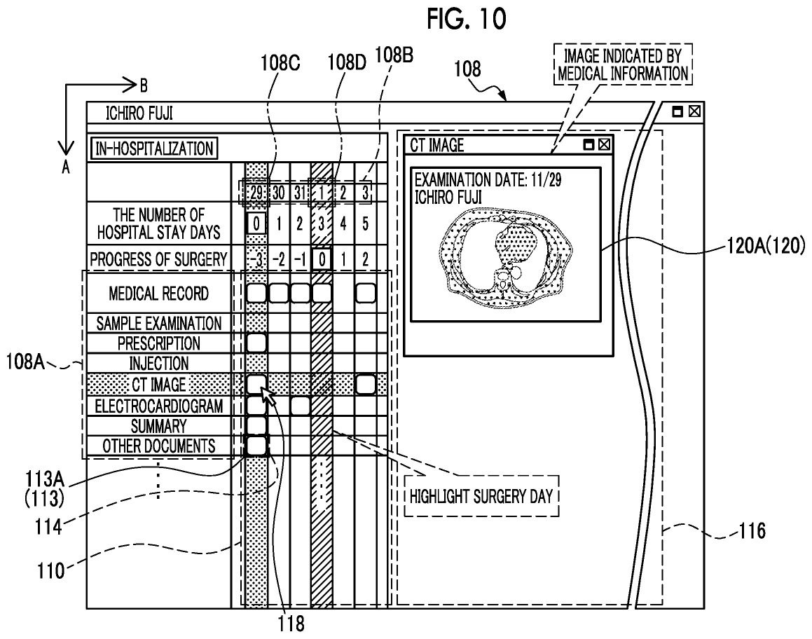

The medical support server 12 is a server that generates a display screen 108 (see FIG. 10) based on information acquired from the electronic medical record server 14, the image management server 16, and the document management server 18, and outputs the generated display screen 108. The electronic medical record server 14 is a server that manages an electronic medical record 94. The image management server 16 is a server that manages image information 102 indicating a plurality of medical images. Examples of the image management server 16 include picture archiving and communication systems (PACS). The document management server 18 is a server that manages document information 106 indicating a plurality of documents. The document refers to, for example, a document created by a medical worker or the like regarding a medical care circumstance for a patient. A first example of the document includes a report created by a doctor, a technician, a pharmacist, and/or a nurse. A second example of the document includes a report generated by the modality. The medical support server 12 is an example of a “medical support device” according to the technology of the present disclosure.

The medical support server 12, the electronic medical record server 14, the image management server 16, and the document management server 18 are connected to each other to be able to communicate with each other via a network 24. Moreover, the medical support server 12 and the client terminal 20 are also connected to each other to be able to communicate with each other via the network 24. Examples of the network 24 include a wide area network (WAN). It should be noted that the WAN is merely an example, and the network 24 may be composed of at least one of a local area network (LAN), a WAN, or the like.

It should be noted that, in an example shown in FIG. 1, the electronic medical record server 14, the image management server 16, and the document management server 18 are connected to the medical support server 12 via the single network 24, but the technology of the present disclosure is not limited to this. For example, the electronic medical record server 14, the image management server 16, and the document management server 18 may be connected to the medical support server 12 via different networks. Moreover, in the example shown in FIG. 1, the client terminal 20 is connected to the medical support server 12 via the single network 24, but the technology of the present disclosure is not limited to this. For example, the client terminal 20 may be connected to the medical support server 12 via a network different from the network to which the electronic medical record server 14, the image management server 16, and the document management server 18 are connected.

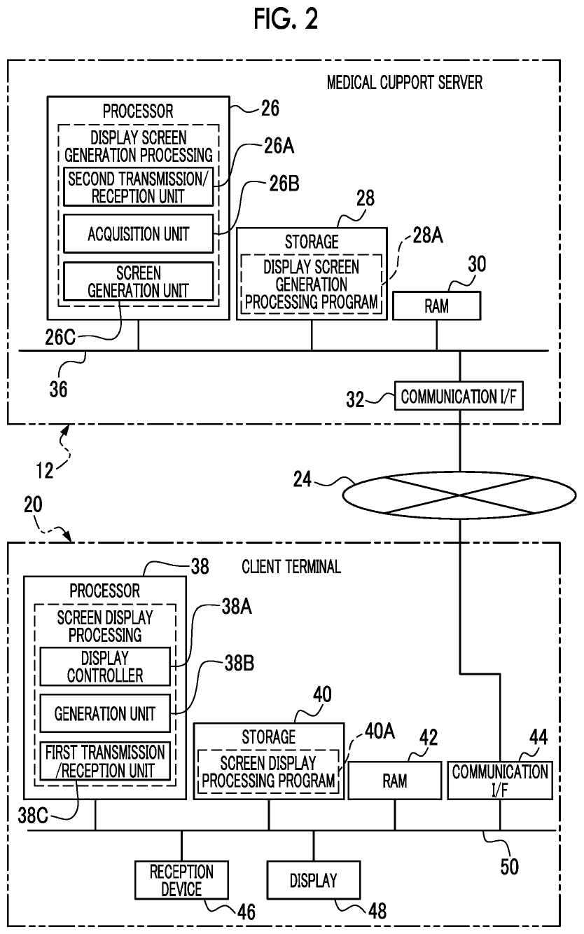

As shown in FIG. 2 as an example, the medical support server 12 comprises a processor 26, a storage 28, a random access memory (RAM) 30, and a communication interface (I/F) 32. The processor 26, the storage 28, the RAM 30, and the communication I/F 32 are connected to a bus 36. It should be noted that the processor 26, the storage 28, and the RAM 30 are examples of a “computer” according to the technology of the present disclosure. Moreover, the processor 26 is an example of a “processor” according to the technology of the present disclosure.

The processor 26 is, for example, a CPU, and controls the entire medical support server 12. Here, the CPU is described as the example of the processor 26, but this is merely an example, and the processor 26 may include a central processing unit (CPU) and a graphics processing unit (GPU). The GPU is operated under the control of the CPU. Moreover, the processor 26 may be one or more CPUs integrated with GPU functions, or may be one or more CPUs which is not integrated with the GPU functions.

A memory is connected to the processor 26. The memory includes the storage 28 and the RAM 30. The storage 28 is a non-volatile storage device that stores various programs, various parameters, and the like. Examples of the storage 28 include a flash memory (for example, an electrically erasable and programmable read only memory (EEPROM) and a solid state drive (SSD)), and a hard disk drive (HDD). It should be noted that the flash memory and the HDD are merely examples, and for example, at least one of the flash memory, the HDD, a magnetoresistive memory, or a ferroelectric memory may be used as the storage 28.

The RAM 30 is a memory in which the information is transitorily stored, and is used as a work memory by the processor 26. Examples of the RAM 30 include a dynamic random access memory (DRAM) and a static random access memory (SRAM).

The communication I/F 32 is connected to the network 24. The client terminal 20 is connected to the network 24, and the communication I/F 32 controls the exchange of the information with the client terminal 20 via the network 24. For example, the communication I/F 32 transmits the information in response to a request from the processor 26 to the client terminal 20 via the network 24. Moreover, the communication I/F 32 receives the information transmitted from the client terminal 20 via the network 24 to output the received information to the processor 26 via the bus 36.

The client terminal 20 comprises a processor 38, a storage 40, a RAM 42, a communication I/F 44, a reception device 46, and a display 48. The processor 38, the storage 40, the RAM 42, the communication I/F 44, the reception device 46, and the display 48 are connected to a bus 50.

The processor 38 controls the entire client terminal 20 in cooperation with the storage 40 and the RAM 42. It should be noted that, here, specific examples of the processor 38, the storage 40, and the RAM 42 are the same as specific examples of the processor 26, the storage 28, and the RAM 30 described above. Therefore, here, the description of the specific examples of the processor 38, the storage 40, and the RAM 42 will be omitted.

The communication I/F 44 is connected to the communication I/F 32 of the medical support server 12 via the network 24, and the communication I/F 44 controls the exchange of information with the medical support server 12 via the network 24. For example, the communication I/F 44 transmits the information in response to a request from the processor 38 to the medical support server 12 via the network 24. Moreover, the communication I/F 44 receives the information transmitted from the medical support server 12 via the network 24 to output the received information to the processor 38 via the bus 50.

The reception device 46 receives an instruction from a user of the client terminal 20. Examples of the user of the client terminal 20 include the medical worker (for example, the doctor, the nurse, and/or the technician). The instruction received by the reception device 46 is acquired by the processor 38. Examples of the reception device 46 include a keyboard and/or a mouse. The keyboard and/or the mouse is merely an example, and at least one of a proximity input device that receives proximity input, a voice input device that receives voice input, or a gesture input device that receives gesture input may be applied as the reception device 46, instead of the keyboard and/or the mouse or together with the keyboard and/or the mouse. The proximity input device is, for example, a touch panel or a tablet. Moreover, the connection between the reception device 46 and the client terminal 20 may be wired or wireless.

The display 48 displays various types of information (for example, an image and a character) under the control of the processor 38. Examples of the display 48 include an electro-luminescence (EL) display and a liquid crystal display. The display 48 is an example of a “display device” according to the technology of the present disclosure.

In the client terminal 20, a screen display processing program 40A is stored in the storage 40. The processor 38 reads out the screen display processing program 40A from the storage 40 and executes the read out screen display processing program 40A on the RAM 42 to perform screen display processing. The screen display processing is realized by operating the processor 38 as a display controller 38A, a generation unit 38B, and a first transmission/reception unit 38C.

In the medical support server 12, a display screen generation processing program 28A is stored in the storage 28. The processor 26 reads out the display screen generation processing program 28A from the storage 28 and executes the read out display screen generation processing program 28A on the RAM 30 to perform display screen generation processing. The display screen generation processing is realized by operating the processor 26 as a second transmission/reception unit 26A, an acquisition unit 26B, and a screen generation unit 26C. It should be noted that the display screen generation processing program 28A is an example of a “program” according to the technology of the present disclosure.

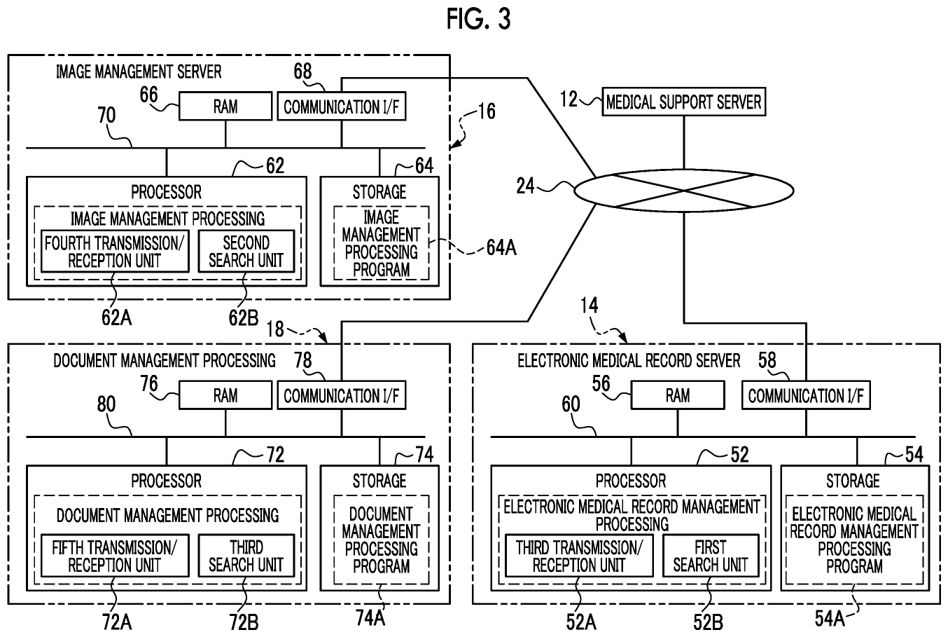

As shown in FIG. 3 as an example, the electronic medical record server 14 comprises a processor 52, a storage 54, a RAM 56, and a communication I/F 58. The processor 52, the storage 54, the RAM 56, and the communication I/F 58 are connected to a bus 60.

The processor 52 controls the entire electronic medical record server 14 in cooperation with the storage 54 and the RAM 56. It should be noted that, here, specific examples of the processor 52, the storage 54, and the RAM 56 are the same as specific examples of the processor 26, the storage 28, and the RAM 30 described above. Therefore, here, the specific examples of the processor 52, the storage 54, and the RAM 56 will be omitted.

The communication I/F 58 is connected to the network 24. The medical support server 12 is connected to the network 24, and the communication I/F 58 controls the exchange of information with the medical support server 12 via the network 24. For example, the communication I/F 58 transmits the information in response to a request from the processor 52 to the medical support server 12 via the network 24. Moreover, the communication I/F 58 receives the information transmitted from the medical support server 12 via the network 24 to output the received information to the processor 52 via the bus 60.

The image management server 16 comprises a processor 62, a storage 64, a RAM 66, and a communication I/F 68. The processor 62, the storage 64, the RAM 66, and the communication I/F 68 are connected to a bus 70.

The processor 62 controls the entire image management server 16 in cooperation with the storage 64 and the RAM 66. It should be noted that, here, specific examples of the processor 62, the storage 64, and the RAM 66 are the same as specific examples of the processor 26, the storage 28, and the RAM 30 described above. Therefore, here, the specific examples of the processor 62, the storage 64, and the RAM 66 will be omitted.

The communication I/F 68 is connected to the network 24. The medical support server 12 is connected to the network 24, and the communication I/F 68 controls the exchange of information with the medical support server 12 via the network 24. For example, the communication I/F 68 transmits the information in response to a request from the processor 62 to the medical support server 12 via the network 24. Moreover, the communication I/F 68 receives the information transmitted from the medical support server 12 via the network 24 to output the received information to the processor 62 via the bus 70.

The document management server 18 comprises a processor 72, a storage 74, a RAM 76, and a communication I/F 78. The processor 72, the storage 74, the RAM 76, and the communication I/F 78 are connected to a bus 80.

The processor 72 controls the entire document management server 18 in cooperation with the storage 74 and the RAM 76. It should be noted that, here, specific examples of the processor 72, the storage 74, and the RAM 76 are the same as specific examples of the processor 26, the storage 28, and the RAM 30 described above. Therefore, here, the specific examples of the processor 72, the storage 74, and the RAM 76 will be omitted.

The communication I/F 78 is connected to the network 24. The medical support server 12 is connected to the network 24, and the communication I/F 78 controls the exchange of information with the medical support server 12 via the network 24. For example, the communication I/F 78 transmits the information in response to a request from the processor 72 to the medical support server 12 via the network 24. Moreover, the communication I/F 78 receives the information transmitted from the medical support server 12 via the network 24 to output the received information to the processor 72 via the bus 80.

In the electronic medical record server 14, an electronic medical record management processing program 54A is stored in the storage 54. The processor 52 reads out the electronic medical record management processing program 54A from the storage 54 and executes the read out electronic medical record management processing program 54A on the RAM 56 to perform electronic medical record management processing. The electronic medical record management processing is realized by operating the processor 52 as a third transmission/reception unit 52A and a first search unit 52B.

In the image management server 16, an image management processing program 64A is stored in the storage 64. The processor 62 reads out the image management processing program 64A from the storage 64 and executes the read out image management processing program 64A on the RAM 66 to perform image management processing. The image management processing is realized by operating the processor 62 as a fourth transmission/reception unit 62A and a second search unit 62B.

In the document management server 18, a document management processing program 74A is stored in the storage 74. The processor 72 reads out the document management processing program 74A from the storage 74 and executes the read out document management processing program 74A on the RAM 76 to perform document management processing. The document management processing is realized by operating the processor 72 as a fifth transmission/reception unit 72A and a third search unit 72B.

By the way, in a case in which the medical worker browses the medical information of the patient (for example, an electronic medical record, an examination report, and/or an examination image), the medical information is displayed on a display of a terminal in time series. As a result, the medical worker can grasp the medical information from the past to the present of the patient over time. As a result, the medical worker can make use of the medical information for planning a future treatment plan and/or selecting a medical practice. Here, in a case in which the medical information is displayed in time series, the medical information may be displayed for the entire period from the past to the present.

However, in a case in which the medical information for the entire period is displayed, it is difficult to grasp the medical information only for a specific period (for example, a hospitalization period). During the hospitalization period, it is required to focus on grasping the medical information as compared with a case in which the patient is not hospitalized because a disease condition is serious or the patient waits for the surgery.

Here, in grasping the medical information limited to the hospitalization period, it is conceivable that the medical worker inputs the hospitalization period by himself/herself, but it is necessary for the medical worker to grasp the hospitalization period in advance in this aspect. However, the medical worker is required to deal with a large number of patients. Therefore, it is difficult for the medical worker to grasp the hospitalization period of each patient in advance. Further, in a case in which the patient has a history of hospitalization a plurality of times, it is necessary to grasp which hospitalization period is the hospitalization period among a plurality of hospitalization periods, and it is more difficult to grasp the hospitalization period.

In view of such circumstances, the medical support server 12 of the medical support system 10 according to the present embodiment performs the display screen generation processing (see FIG. 2). Moreover, the client terminal 20 performs the screen display processing (see FIG. 2).

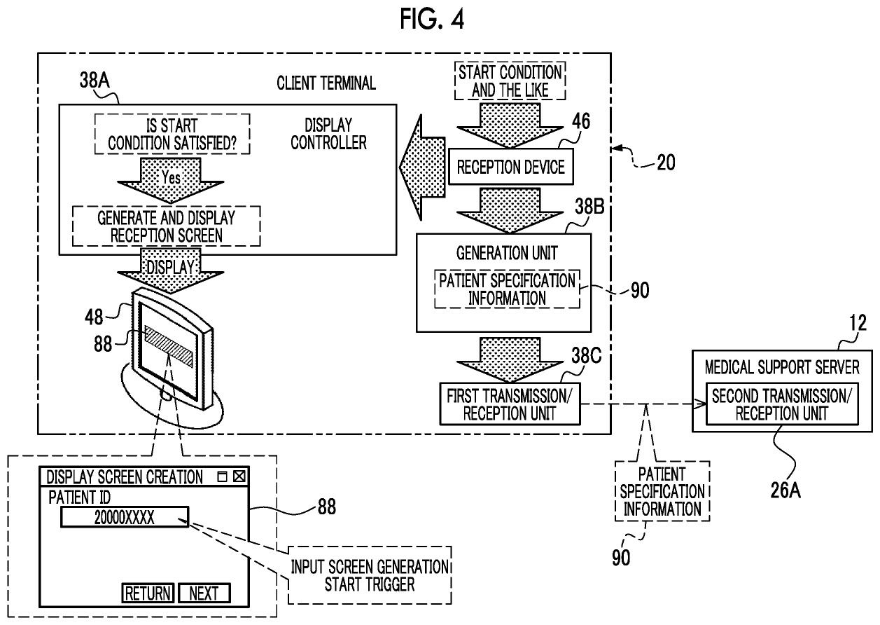

As shown in FIG. 4 as an example, in the client terminal 20, the display controller 38A determines whether or not a condition for causing the medical support server 12 to start the generation of the display screen 108 (see FIG. 10) (hereinafter, also referred to as “start condition”) is satisfied. Examples of the start condition include a condition that the reception device 46 receives an instruction to cause the medical support server 12 to start the generation of the display screen 108 (see FIG. 10).

In a case in which the start condition is satisfied, the display controller 38A generates a reception screen 88 and displays the reception screen 88 on the display 48. A trigger (hereinafter, also referred to as a “screen generation start trigger”) for the medical support server 12 to start the generation of the display screen 108 (see FIG. 10) is input to the reception screen 88 in response to the instruction received by the reception device 46. In an example shown in FIG. 4, an example is shown in which a patient identification data (ID) is input as the screen generation start trigger to the reception screen 88, but this is merely an example. A name of the patient (hereinafter, also referred to as “patient name”) may be input to the reception screen 88 together with the patient ID or instead of the patient ID. Moreover, the screen generation start trigger may be received via a screen on which the electronic medical record 94 of the patient is displayed.

The generation unit 38B generates patient specification information 90. The patient specification information 90 refers to information for specifying the patient. Here, for example, the patient ID received as the screen generation start trigger by the reception device 46 is applied as the patient specification information 90. The first transmission/reception unit 38C transmits the patient specification information 90 generated by the generation unit 38B to the medical support server 12. In the medical support server 12, the second transmission/reception unit 26A receives the patient specification information 90.

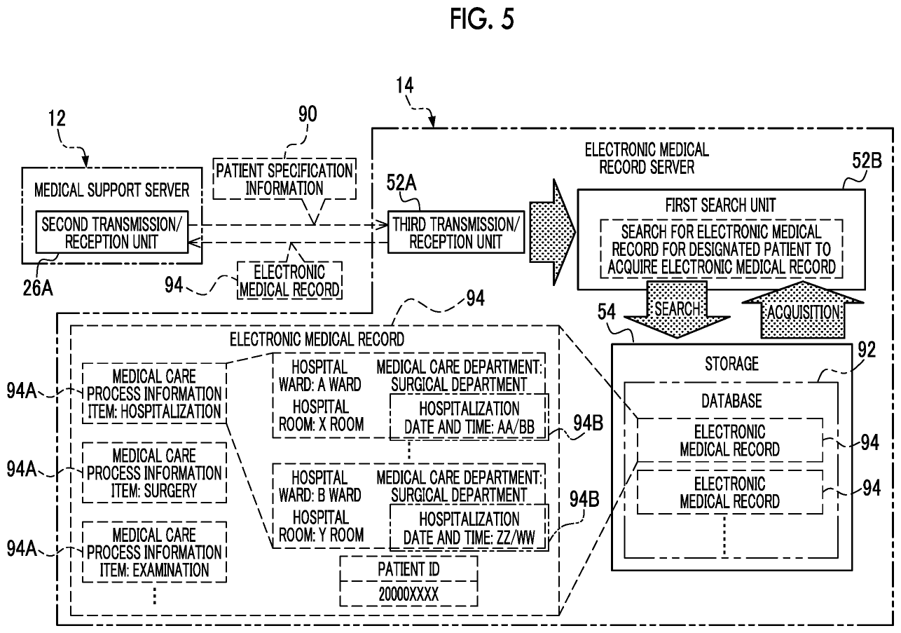

As shown in FIG. 5 as an example, the second transmission/reception unit 26A transmits the received patient specification information 90 to the electronic medical record server 14. In a case in which the patient specification information 90 is transmitted from the second transmission/reception unit 26A of the medical support server 12 to the electronic medical record server 14, the third transmission/reception unit 52A of the electronic medical record server 14 receives the patient specification information 90.

In the storage 54 of the electronic medical record server 14, a database 92 is constructed. A plurality of electronic medical records 94 are stored in the database 92. Each of the plurality of electronic medical records 94 is, for example, information peculiar to each patient. Medical care process information 94A is included in each of the plurality of electronic medical records 94. The medical care process information 94A is information indicating a medical care record obtained for each medical care process (hereinafter, also referred to as “medical care record”). In an example shown in FIG. 5, a medical care record of the hospitalization, a medical care record of the surgery, and a medical care record of the examination are shown as the medical care process information 94A. As the medical care process information 94A, the medical care record of the hospitalization includes date information 94B indicating a hospitalization day and a hospital discharge day. Here, the date information refers to information indicating a date.

The first search unit 52B searches the database 92 to acquire the electronic medical record 94 in accordance with the patient specification information 90 received by the third transmission/reception unit 52A. In this case, for example, the first search unit 52B searches the database 92 to acquire the electronic medical record 94 in which information that coincides with the patient specification information 90 (here, as an example, the patient ID) is included.

In the electronic medical record server 14, the third transmission/reception unit 52A transmits the electronic medical record 94 acquired by the first search unit 52B to the medical support server 12. In the medical support server 12, the second transmission/reception unit 26A receives the electronic medical record 94 transmitted from the electronic medical record server 14.

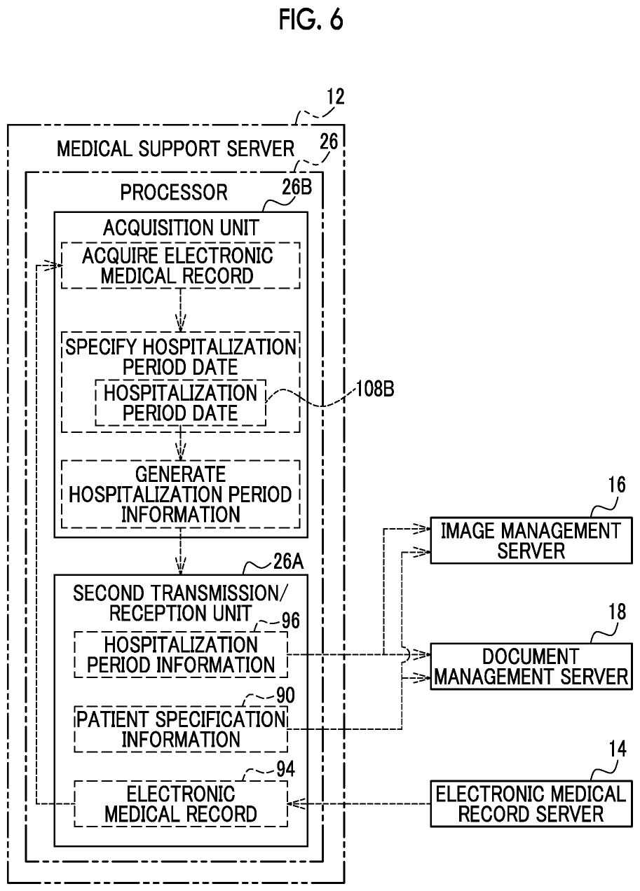

As shown in FIG. 6 as an example, the acquisition unit 26B acquires the electronic medical record 94 received by the second transmission/reception unit 26A. The acquisition unit 26B acquires a hospitalization period date 108B (see FIG. 10) based on the acquired electronic medical record 94. Stated another way, the acquisition unit 26B specifies the hospitalization period date 108B corresponding to the hospitalization period. Here, the hospitalization period date 108B refers to a date of a period in which the patient is hospitalized or has been hospitalized. Moreover, the specification of the hospitalization period date 108B refers to processing of calculating the period in which the patient has been hospitalized from the dates of the hospitalization day and the hospital discharge day specified from the electronic medical record 94. For example, the hospitalization day is specified based on the date information 94B included in the medical care process information 94A (see FIG. 5) related to the hospitalization. Moreover, similarly, the hospital discharge day is specified based on the date information 94B included in the medical care process information 94A (see FIG. 5) related to the hospital discharge. Here, the hospital discharge day may be a scheduled hospital discharge day instead of an actual hospital discharge day, or may be a day on which a predetermined period (for example, one week) has elapsed from the hospitalization day. Moreover, in a case in which the hospital discharge day is not input, a date of a day on which the display screen 108 is created may be used instead of the hospital discharge day.

Moreover, the hospitalization period date 108B is not always fixed and may be changed. For example, the hospitalization period date 108B may be an aspect in which the date is extended each time the hospitalization period is extended.

The acquisition unit 26B generates hospitalization period information 96 indicating the hospitalization period date 108B. It should be noted that the hospitalization period date 108B is an example of a “hospitalization period date” according to the technology of the present disclosure.

The second transmission/reception unit 26A of the medical support server 12 transmits the hospitalization period information 96 and the patient specification information 90 to the image management server 16 and the document management server 18.

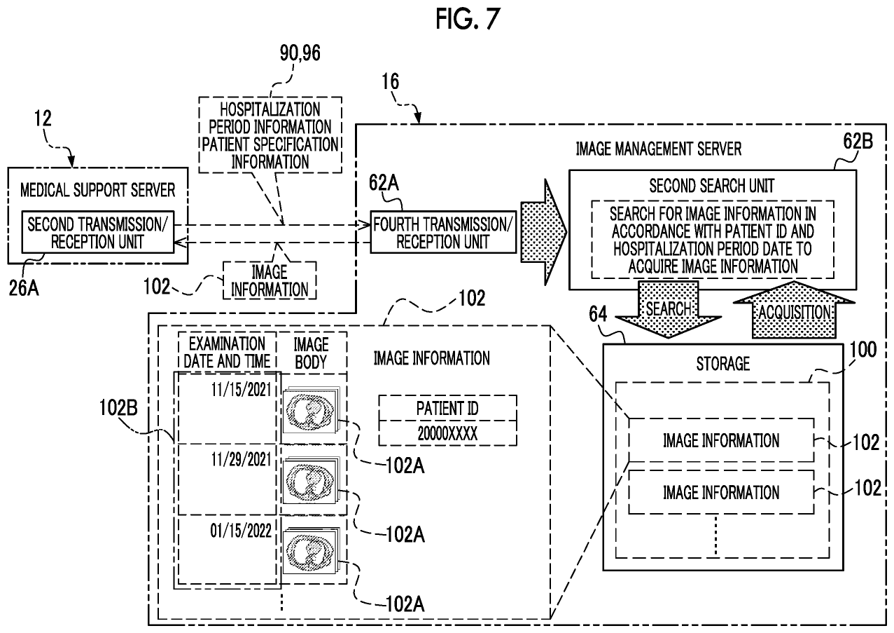

As shown in FIG. 7 as an example, the fourth transmission/reception unit 62A of the image management server 16 receives the hospitalization period information 96 and the patient specification information 90. In the storage 64 of the image management server 16, a database 100 is constructed. A plurality of pieces of image information 102 are stored in the database 100. Each of the plurality of pieces of image information 102 is, for example, image information peculiar to each patient. The image information 102 is information indicating an image obtained by examination using various modalities. The image information 102 includes the patient ID, image body information 102A, and image date information 102B. The image body information 102A is information indicating the image obtained from the modality. The image date information 102B is information indicating date and time when the examination is performed. It should be noted that, here, the information indicating the date and time when the examination is performed has been described as the image date information 102B, but this is merely an example. For example, information indicating date and time when the image information 102 is created may be applied as the image date information 102B.

The second search unit 62B searches the database 100 to acquire the image information 102 in accordance with the patient ID indicated by the patient specification information 90 received by the fourth transmission/reception unit 62A and the hospitalization period date 108B (see FIG. 6) indicated by the hospitalization period information 96 received by the fourth transmission/reception unit 62A. In this case, for example, the second search unit 62B searches the database 100 to acquire the image information 102 in which the patient ID indicated by the patient specification information 90 and the image date information 102B indicating the date that coincides with the hospitalization period date 108B indicated by the hospitalization period information 96 are included.

In the image management server 16, the fourth transmission/reception unit 62A transmits the image information 102 acquired by the second search unit 62B to the medical support server 12. In the medical support server 12, the second transmission/reception unit 26A receives the image information 102 transmitted from the image management server 16.

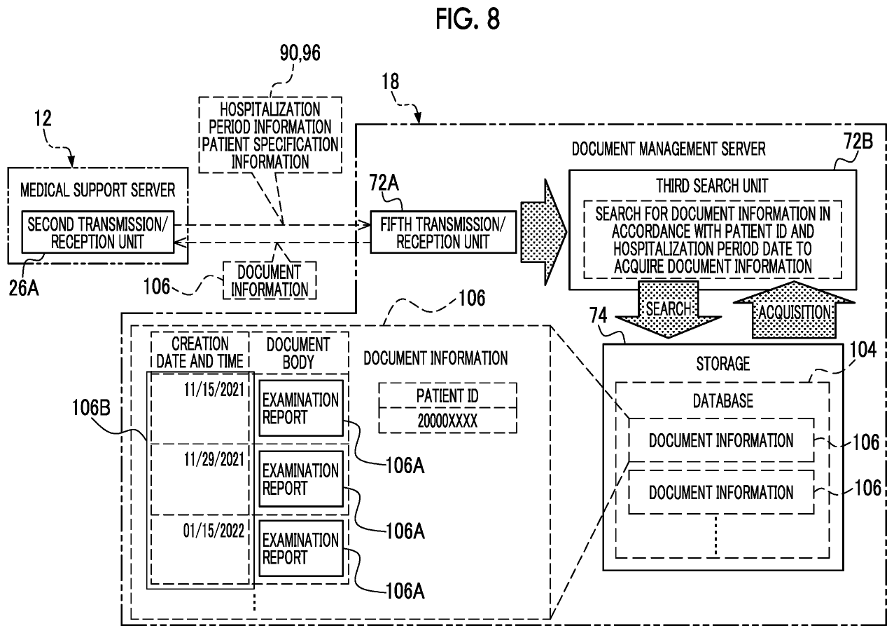

As shown in FIG. 8 as an example, the fifth transmission/reception unit 72A of the document management server 18 receives the hospitalization period information 96 and the patient specification information 90. In the storage 74 of the document management server 18, a database 104 is constructed. A plurality of pieces of document information 106 are stored in the database 104. Each of the plurality of pieces of document information 106 is, for example, document information peculiar to each patient. The document information 106 is information indicating a document created by the medical worker. The document information 106 includes the patient ID, document body information 106A, and document date information 106B. The document body information 106A is information indicating a document body. The document date information 106B is information indicating date and time when the document is created. It should be noted that, here, the information indicating the date and time when the document is created has been described as the document date information 106B, but this is merely an example. For example, information indicating date and time when the document information 106 is created may be applied as the document date information 106B.

The third search unit 72B searches the database 104 to acquire the document information 106 in accordance with the patient ID indicated by the patient specification information 90 received by the fifth transmission/reception unit 72A and the hospitalization period date 108B (see FIG. 6) indicated by the hospitalization period information 96. That is, the third search unit 72B searches the database 104 to acquire the document information 106 in which the patient ID indicated by the patient specification information 90 and the document date information 106B that coincides with the hospitalization period date 108B indicated by the hospitalization period information 96 are included.

In the document management server 18, the fifth transmission/reception unit 72A transmits the document information 106 acquired by the third search unit 72B to the medical support server 12. In the medical support server 12, the second transmission/reception unit 26A receives the document information 106 transmitted from the document management server 18.

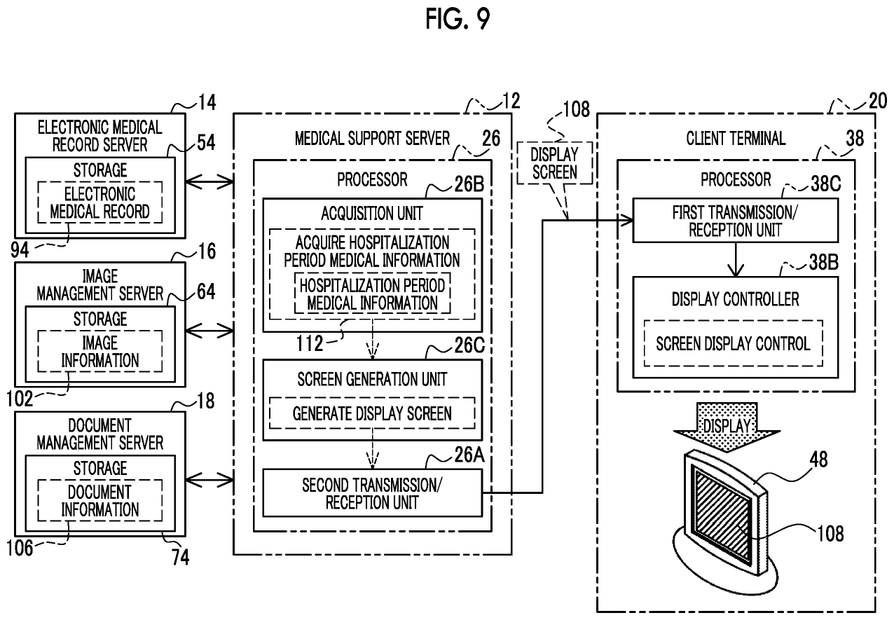

As shown in FIG. 9 as an example, the acquisition unit 26B acquires the image information 102 in accordance with the hospitalization period date 108B from the image management server 16. Specifically, the acquisition unit 26B acquires the image information 102 in which the image date information 102B (see FIG. 7) indicating the hospitalization period date 108B is included from the image management server 16. Moreover, the acquisition unit 26B acquires the document information 106 in accordance with the hospitalization period date 108B from the document management server 18. Specifically, the acquisition unit 26B acquires the document information 106 in which the document date information 106B (see FIG. 8) indicating the hospitalization period date 108B is included from the document management server 18.

Here, the electronic medical record 94, the image information 102, and the document information 106 acquired by the acquisition unit 26B include a plurality of items 108A (see FIG. 10). For example, the electronic medical record 94 includes an item “electronic medical record article”, and the image information 102 includes an item related to various images. Here, the various images refer to, for example, images obtained from various modalities. Examples of the images obtained from various modalities include a computed tomography (CT) image and/or a magnetic resonance imaging (MRI) image. Moreover, the document information 106 includes items, such as a summary and/or other documents. Moreover, as described above, the date information is included in the electronic medical record 94, the image information 102, and the document information 106. That is, the plurality of items 108A (see FIG. 10) included in the electronic medical record 94, the image information 102, and the document information 106 acquired by the acquisition unit 26B are associated with the hospitalization period date 108B indicated by the date information. That is, the acquisition unit 26B acquires hospitalization period medical information 112 including the plurality of items 108A associated with the hospitalization period date 108B. It should be noted that the plurality of items 108A are an example of a “plurality of first items” according to the technology of the present disclosure, and the hospitalization period medical information 112 is an example of “hospitalization period medical information” according to the technology of the present disclosure.

The screen generation unit 26C generates the display screen 108 (see FIG. 10) based on the hospitalization period medical information 112 acquired by the acquisition unit 26B. That is, the screen generation unit 26C generates the display screen 108 on which the hospitalization period medical information 112 is disposed in a matrix in a region in which the hospitalization period date 108B is on a horizontal axis B (see FIG. 10), and the plurality of items 108A (see FIG. 10) included in the electronic medical record 94, the image information 102, and the document information 106 are on a vertical axis A (see FIG. 10).

The second transmission/reception unit 26A transmits the information indicating the display screen 108 (see FIG. 10) generated by the screen generation unit 26C to the client terminal 20.

In the client terminal 20, the first transmission/reception unit 38C receives the information indicating the display screen 108 (see FIG. 10). The display controller 38A displays the display screen 108 on the display 48 based on the information indicating the display screen 108 received by the first transmission/reception unit 38C.

As shown in FIG. 10 as an example, the display screen 108 includes a first display region 110. The first display region 110 is a region defined by the vertical axis A and the horizontal axis B. The vertical axis A refers to an axis indicating the plurality of items 108A included in the hospitalization period medical information 112. The horizontal axis B refers to an axis indicating the hospitalization period date 108B. In an example shown in FIG. 10, a medical record, a sample examination, a prescription, an injection, the CT image, an electrocardiogram, the summary, and other documents are displayed as examples of the plurality of items 108A on the vertical axis A. Moreover, as an example of the hospitalization period date 108B on the horizontal axis B, a date from the 29th to the 31st of a certain month and a date from the 1st to the 3rd of the following month are displayed. It should be noted that the vertical axis A is an example of a “second axis” according to the technology of the present disclosure, the horizontal axis B is an example of a “first axis” according to the technology of the present disclosure, the first display region 110 is an example of a “first display region” according to the technology of the present disclosure, and the display screen 108 is an example of a “display screen” according to the technology of the present disclosure.

In the example shown in FIG. 10, the medical record, the sample examination, the prescription, the injection, the CT image, the electrocardiogram, the summary, and other documents are displayed as the plurality of items on the vertical axis A, these are merely examples. For example, item examples of biopsy examination among the plurality of items on the vertical axis A include general imaging, mammography, MRI examination, X-ray angiography, nuclear medicine, positron emission tomography (PET) examination, and ultrasonic examination. Moreover, item examples of the examination images include an otolaryngology examination image, an ophthalmology examination image, and a digital camera image. Moreover, item examples of a medical care document and a scan document include a medical care plan, a hospital discharge summary, a nursing summary, a medical care information provision form, a letter of introduction, a surgery record, a home-visit nursing record, an examination consent form, a surgery consent form, an anesthesia consent form, a blood transfusion consent form, a clinical trial consent form, a medical certificate, and a certificate.

The plurality of items on the vertical axis A may be predetermined items (for example, items determined for each user and/or for each medical facility) or may be set by the user. In a case in which the items are set by the user, the items on the vertical axis A are received via the reception device 46.

The hospitalization period medical information 112 is disposed in a matrix in the first display region 110. Here, the fact that the hospitalization period medical information 112 is disposed in a matrix includes an aspect in which information indicating the presence, a state, or a type of the medical information 120 corresponding to the hospitalization period medical information 112 is disposed, in addition to an aspect in which the hospitalization period medical information 112 itself is disposed. In the example shown in FIG. 10, quadrangular icons 113 indicating the presence of the medical information 120 are disposed in a matrix in the first display region 110. In the example shown in FIG. 10, an icon 113A is displayed in a cell 114 at a position at which a row of the corresponding item 108A among the plurality of items 108A intersects with a column of the corresponding date among the hospitalization period dates 108B. That is, the icon 113 indicates the presence of the medical information 120 corresponding to the cell 114. The icon 113 is an example of an “icon” according to the technology of the present disclosure, and the cell 114 is an example of a “cell” according to the technology of the present disclosure.

On the display screen 108, the number of days (that is, the number of hospital stay days) based on a hospitalization day 108C is displayed. In the example shown in FIG. 10, the number of hospital stay days is displayed in a row below the row in which the hospitalization period date 108B is displayed (that is, a next row along a direction of the vertical axis A). Here, the hospitalization day 108C includes a date of scheduled hospitalization, in addition to a date of actual hospitalization. In the example shown in FIG. 10, the 0th to 5th days, which are the number of days based on the 29th, are displayed as the number of hospital stay days. That is, the number of days based on the hospitalization day 108C is displayed for all the dates in the hospitalization period. It should be noted that, in the example shown in FIG. 10, the example has been described in which a plurality of dates are included in the hospitalization period date 108B and the number of hospital stay days is displayed for each of all the dates included in the hospitalization period date 108B, but this is merely an example. The number of hospital stay days may be displayed only on a part of the dates included in the hospitalization period date 108B in a case in which a plurality of dates are included in the hospitalization period date 108B. It should be noted that the hospitalization period date 108B may be a single date. It should be noted that the hospitalization day 108C is an example of a “hospitalization day” according to the technology of the present disclosure.

On the display screen 108, the number of days (that is, the progress of the surgery) based on a surgery day 108D is displayed. In the example shown in FIG. 10, the progress of the surgery is displayed in a row below the row in which the number of hospital stay days is displayed (that is, a next row along the direction of the vertical axis A). Here, the surgery day 108D includes a date on which the surgery is scheduled, in addition to a date on which the surgery is actually performed. The surgery day 108D is specified, for example, based on the medical care process information 94A related to the surgery included in the electronic medical record 94.

In the example shown in FIG. 10, as the progress of the surgery, the −3rd to the 2nd days (that is, +2nd day), which are the number of days based on the 1st, which is the surgery day 108D, are displayed. It should be noted that, in the example shown in FIG. 10, the example has been described in which a plurality of dates are included in the hospitalization period date 108B and the progress of the surgery is further displayed for each of all the dates included in the hospitalization period date 108B, but this is merely an example. The progress of the surgery may be displayed only on a part of the dates included in the hospitalization period date 108B in a case in which a plurality of dates are included in the hospitalization period date 108B. It should be noted that the surgery day 108D is an example of a “surgery day” according to the technology of the present disclosure.

In the example shown in FIG. 10, a column of the date of the 1st which is the surgery day 108D is colored differently from other columns, so that the column of the date of the surgery day 108D is visually highlighted. As a result, on the display screen 108, the surgery day 108D can be distinguished from a date other than the surgery day 108D. It should be noted that, in the example shown in FIG. 10, the example has been described in which the column of the surgery day 108D is colored differently from other columns, so that the surgery day 108D and a date other than the surgery day 108D are displayed to be distinguishable, but this is merely an example. For example, a special pattern may be applied to the column of the surgery day 108D to enable distinguishing the column of the surgery day 108D from other columns, and a character color, a character size, and/or a font of the date indicating the surgery day 108D may be displayed in a color different from those of other columns to make it possible to distinguish the column of the surgery day 108D from other columns.

In the example shown in FIG. 10, the icon 113 is designated via a pointer 118 on the display screen 108. The date on which the designated icon 113 is specified (that is, a date corresponding to the designated icon 113 on the horizontal axis B of the display screen 108) is displayed to be distinguishable from other dates. Further, the item 108A for specifying the designated icon 113 (that is, the item 108A corresponding to the designated icon 113 on the vertical axis A of the display screen 108) is displayed to be distinguishable from other items 108A. In the example shown in FIG. 10, a column corresponding to the 29th, which is the date for specifying the designated icon 113, and a row corresponding to the item 108A of the CT image, which is the item 108A for specifying the designated icon 113, are highlighted.

The display screen 108 includes a medical information display region 116. The medical information 120 is displayed in the medical information display region 116. The medical information 120 is information specified by the item 108A. In a case in which the icon 113 is designated via the pointer 118 on the display screen 108, the medical information 120 corresponding to the icon 113 is displayed in the medical information display region 116. In the example shown in FIG. 10, in the first display region 110, the icon 113 is displayed in the cell 114 specified by the date of the 29th and the item 108A of the CT image. In a case in which the icon 113 is designated via the pointer 118, a CT image 120A, which is an example of the medical information 120 corresponding to the icon 113, is displayed in the medical information display region 116. The medical information display region 116 is an example of a “medical information display region” according to the technology of the present disclosure.

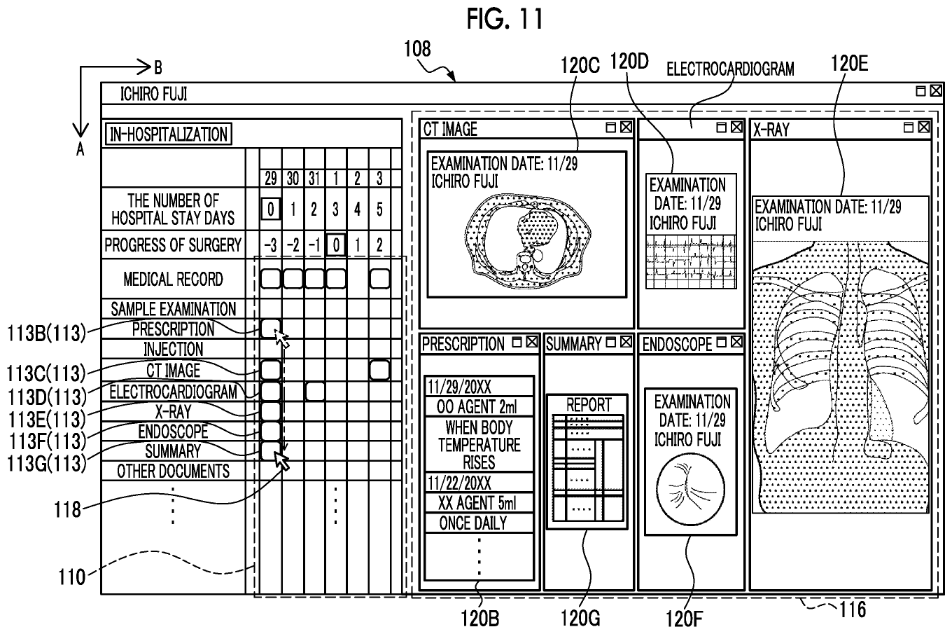

As shown in FIG. 11 as an example, in a case in which a plurality of icons 113 are designated via the pointer 118, a content indicated by the medical information 120 corresponding to each of the plurality of icons 113 is displayed in the medical information display region 116. In an example shown in FIG. 11, a plurality of icons 113B to 113G displayed in a column of the date of the 29th are designated via the pointer 118. In the medical information display region 116, contents indicated by the medical information 120 corresponding to the icons 113B to 113G are displayed. That is, a prescription 120B which is an example of the content indicated by the medical information 120 corresponding to the icon 113B, a CT image 120C which is an example of the content indicated by the medical information 120 corresponding to the icon 113C, and an electrocardiogram 120D which is an example of the content indicated by the medical information 120 corresponding to the icon 113D are displayed in the medical information display region 116. Moreover, an X-ray image 120E which is an example of the content indicated by the medical information 120 corresponding to the icon 113E, an endoscopic image 120F which is an example of the content indicated by the medical information 120 corresponding to the icon 113F, and a summary 120G which is an example of the content indicated by the medical information 120 corresponding to the icon 113G are displayed.

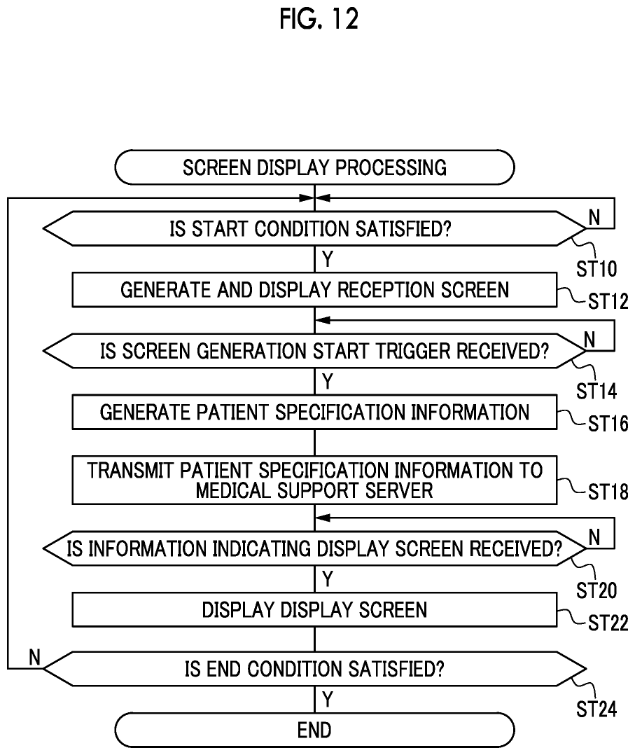

Next, an action of the medical support system 10 will be described with reference to FIGS. 12 to 16.

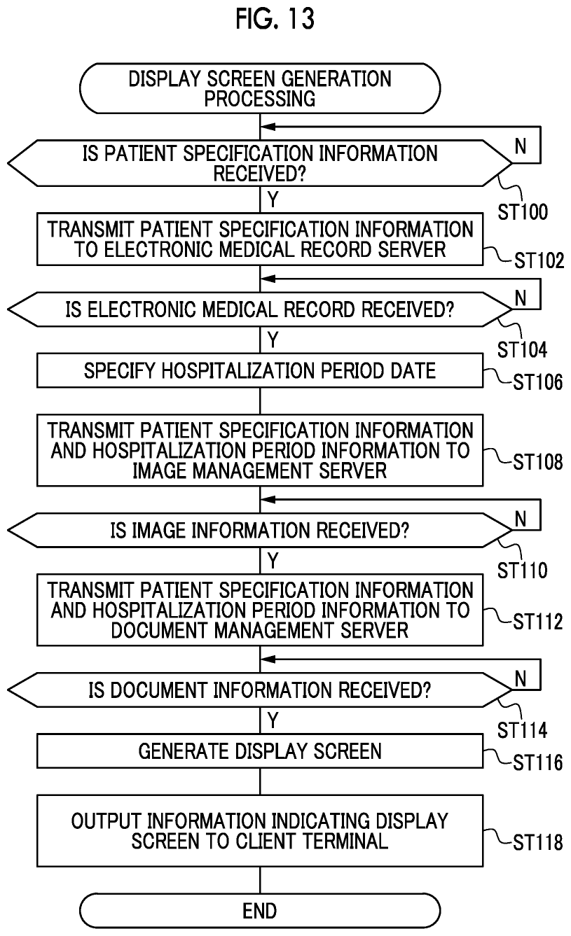

FIG. 12 shows an example of a flow of the screen display processing performed by the processor 38 of the client terminal 20. FIG. 13 shows an example of a flow of the display screen generation processing performed by the processor 26 of the medical support server 12.

First, an example of a flow of the screen display processing performed by the processor 38 of the client terminal 20 will be described with reference to FIG. 12.

In the screen display processing shown in FIG. 12, first, in step ST10, the display controller 38A determines whether or not the start condition is satisfied. In step ST10, in a case in which the start condition is not satisfied, a negative determination is made, and the screen display processing shifts to step ST10. In step ST10, in a case in which the start condition is satisfied, a positive determination is made, and the screen display processing shifts to step ST12.

In step ST12, the display controller 38A generates the reception screen 88 and displays the generated reception screen 88 on the display 48 (see FIG. 4). After processing of step ST12 is executed, the screen display processing shifts to step ST14.

In step ST14, the display controller 38A determines whether or not the screen generation start trigger is received by the reception device 46. In step ST14, in a case in which the screen generation start trigger is not received by the reception device 46, the screen display processing shifts to step ST14. In step ST14, in a case in which the screen generation start trigger is received by the reception device 46, the screen display processing shifts to step ST16.

In step ST16, the generation unit 38B generates the patient specification information 90 based on the patient ID received by the reception device 46 in step ST14 (see FIG. 4). After the processing of step ST16 is executed, the screen display processing shifts to step ST18.

In step ST18, the first transmission/reception unit 38C transmits the patient specification information 90 generated in step ST18 to the medical support server 12 (see FIG. 4). After the processing of step ST18 is executed, the screen display processing shifts to step ST20.

In a case in which the patient specification information 90 is transmitted from the client terminal 20 to the medical support server 12 by executing the processing of step ST18, the processing of step ST100 to step ST118 included in the display screen generation processing shown in FIG. 13 is executed. As a result, the information indicating the display screen 108 is transmitted from the medical support server 12 to the client terminal 20.

Then, in step ST20, the first transmission/reception unit 38C determines whether or not the information indicating the display screen 108 (see FIG. 10) transmitted from the medical support server 12 is received. In step ST20, in a case in which the information indicating the display screen 108 transmitted from the medical support server 12 is not received, a negative determination is made, and the determination in step ST20 is made again. In step ST20, in a case in which the information indicating the display screen 108 transmitted from the medical support server 12 is received, a positive determination is made, and the screen display processing shifts to step ST22.

In step ST22, the display controller 38A displays the display screen 108 on the display 48 based on the information indicating the display screen 108 received by the communication I/F 44 in step ST20 (see FIG. 9). After the processing of step ST22 is executed, the screen display processing shifts to step ST24.

In step ST24, the processor 38 determines whether or not a condition that the screen display processing ends (hereinafter, referred to as “end condition”) is satisfied. Examples of the end condition include a condition that an instruction to end the screen display processing is received by the reception device 46. In step ST24, in a case in which the end condition is not satisfied, a negative determination is made, and the screen display processing shifts to step ST10. In step ST24, in a case in which the end condition is satisfied, a positive determination is made, and the screen display processing ends.

Next, an example of a flow of the display screen generation processing performed by the processor 26 of the medical support server 12 will be described with reference to FIG. 13. It should be noted that the display screen generation processing shown in FIG. 13 is an example of a “medical support method” according to the technology of the present disclosure.

In the display screen generation processing shown in FIG. 13, first, in step ST100, the second transmission/reception unit 26A determines whether or not the patient specification information 90 transmitted from the client terminal 20 by executing the processing of step ST18 included in the screen display processing shown in FIG. 12 is received by the communication I/F 32 (see FIG. 2). In step ST100, in a case in which the patient specification information 90 is not received by the communication I/F 32, a negative determination is made, and the display screen generation processing shifts to step ST100. In step ST100, in a case in which the patient specification information 90 is received by the communication I/F 32, a positive determination is made, and the display screen generation processing shifts to step ST102.

In step ST102, the second transmission/reception unit 26A transmits the patient specification information 90 received by the communication I/F 32 in step ST100 to the electronic medical record server 14 (see FIG. 5). After the processing of step ST102 is executed, the display screen generation processing shifts to step ST104.

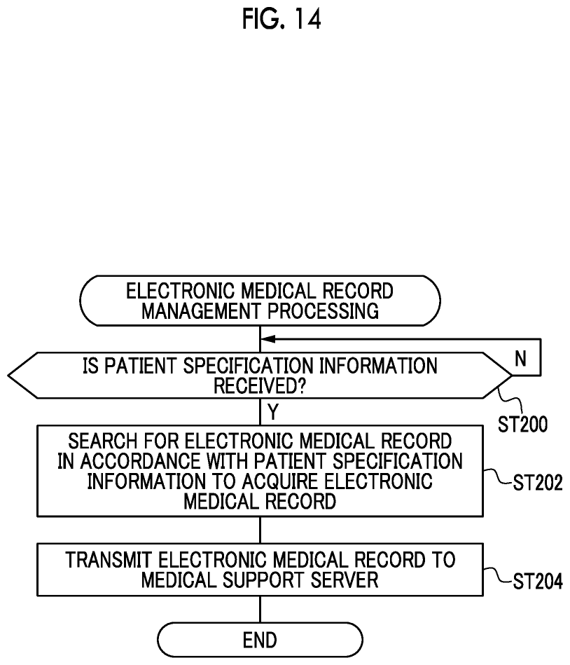

In a case in which the patient specification information 90 is transmitted from the medical support server 12 to the electronic medical record server 14 by executing the processing of step ST104, the processing of step ST200 to step ST204 included in the electronic medical record management processing shown in FIG. 14 is executed, so that the electronic medical record 94 is transmitted from the electronic medical record server 14 to the medical support server 12 (see FIG. 6).

Then, in step ST104, the acquisition unit 26B determines whether or not the electronic medical record 94 transmitted from the electronic medical record server 14 is received by the communication I/F 32 (see FIG. 2). In step ST104, in a case in which the electronic medical record 94 transmitted from the electronic medical record server 14 is not received by the communication I/F 32, a negative determination is made, and the display screen generation processing shifts to step ST102. In step ST104, in a case in which the electronic medical record 94 transmitted from the electronic medical record server 14 is received by the communication I/F 32, a positive determination is made, and the display screen generation processing shifts to step ST106.

In step ST106, the acquisition unit 26B specifies the hospitalization period date 108B based on the electronic medical record 94 acquired in step ST104. After the processing of step ST106 is executed, the display screen generation processing shifts to step ST108.

In step ST108, the acquisition unit 26B transmits the hospitalization period information 96 which is information indicating the hospitalization period date 108B specified in step ST106 to the image management server 16. Moreover, the acquisition unit 26B transmits the patient specification information 90 to the image management server 16. After the processing of step ST108 is executed, the display screen generation processing shifts to step ST110.

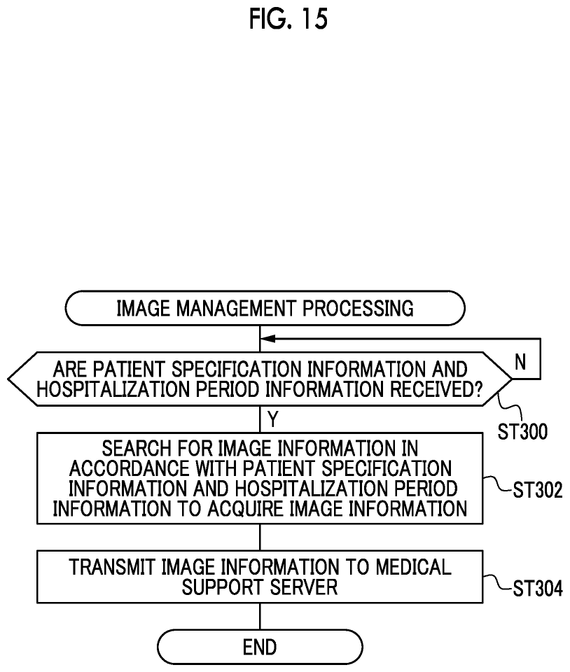

In a case in which the hospitalization period information 96 and the patient specification information 90 are transmitted from the medical support server 12 to the image management server 16 by executing the processing of step ST108, the processing of step ST300 to step ST304 included in the image management processing shown in FIG. 15 is executed, so that the image information 102 is transmitted from the image management server 16 to the medical support server 12 (see FIG. 7).

Then, in step ST110, the acquisition unit 26B determines whether or not the image information 102 transmitted from the image management server 16 is received by the communication I/F 32 (see FIG. 2). In step ST110, in a case in which the image information 102 transmitted from the image management server 16 is not received by the communication I/F 32, a negative determination is made, and the display screen generation processing shifts to step ST110. In step ST110, in a case in which the image information 102 transmitted from the image management server 16 is received by the communication I/F 32, a positive determination is made, and the display screen generation processing shifts to step ST112.

In step ST112, the acquisition unit 26B transmits the hospitalization period information 96 which is information indicating the hospitalization period date 108B specified in step ST106 to the document management server 18. Moreover, the acquisition unit 26B transmits the patient specification information 90 to the document management server 18. After the processing of step ST108 is executed, the display screen generation processing shifts to step ST114.

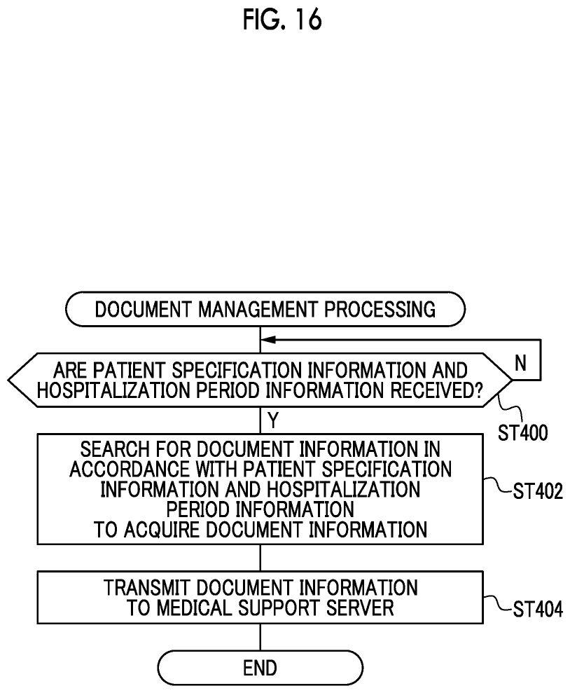

In a case in which the hospitalization period information 96 and the patient specification information 90 are transmitted from the medical support server 12 to the document management server 18 by executing the processing of step ST112, the processing of step ST400 to step ST404 included in the document management processing shown in FIG. 16 is executed, so that the document information 106 is transmitted from the document management server 18 to the medical support server 12 (see FIG. 7).

Then, in step ST114, the acquisition unit 26B determines whether or not the document information 106 transmitted from the document management server 18 is received by the communication I/F 32 (see FIG. 2). In step ST114, in a case in which the document information 106 transmitted from the document management server 18 is not received by the communication I/F 32, a negative determination is made, and the display screen generation processing shifts to step ST110. In step ST114, in a case in which the document information 106 transmitted from the document management server 18 is received by the communication I/F 32, a positive determination is made, and the display screen generation processing shifts to step ST116. It should be noted that, in an example shown in FIG. 13, the form example in which the processing of step ST108 and the processing of step ST112 are performed in an order is given, but the technology of the present disclosure is not limited to this, and the processing may be performed in parallel or may be performed in different order.

In step ST116, the screen generation unit 26C generates the display screen 108 based on the electronic medical record 94, the image information 102, and the document information 106 acquired by the acquisition unit 26B. After the processing of step ST116 is executed, the display screen generation processing shifts to step ST118.

In step ST118, the second transmission/reception unit 26A transmits the information indicating the display screen 108 generated by the screen generation unit 26C to the client terminal 20. After the processing of step ST118 is executed, the screen generation processing ends.

Next, an example of a flow of the electronic medical record management processing performed by the processor 52 of the electronic medical record server 14 will be described with reference to FIG. 14.

In the electronic medical record management processing shown in FIG. 14, first, in step ST200, the third transmission/reception unit 52A determines whether or not the patient specification information 90 transmitted from the medical support server 12 by executing the processing of step ST102 included in the display screen generation processing shown in FIG. 13 is received by the communication I/F 58 (see FIG. 3). In step ST200, in a case in which the patient specification information 90 is not received by the communication I/F 58, a negative determination is made, and the electronic medical record management processing shifts to step ST200. In step ST200, in a case in which the patient specification information 90 is received by the communication I/F 58, a positive determination is made, and the electronic medical record management processing shifts to step ST202.

In step ST202, the first search unit 52B searches the database 92 (see FIG. 5) to acquire the electronic medical record 94 (see FIG. 5) in accordance with the patient ID indicated by the patient specification information 90 received by the communication I/F 58 in step ST200. After the processing of step ST202 is executed, the electronic medical record management processing shifts to step ST204.

In step ST204, the third transmission/reception unit 52A transmits the electronic medical record 94 acquired in step ST202 to the medical support server 12. After the processing of step ST204 is executed, the electronic medical record management processing ends.

Next, an example of a flow of the image management processing performed by the processor 62 of the image management server 16 will be described with reference to FIG. 15.

In the image management processing shown in FIG. 15, first, in step ST300, the fourth transmission/reception unit 62A determines whether or not the patient specification information 90 and the hospitalization period information 96 transmitted from the medical support server 12 by executing the processing of step ST108 included in the display screen generation processing shown in FIG. 13 are received by the communication I/F 68 (see FIG. 3). In step ST300, in a case in which the patient specification information 90 and the hospitalization period information 96 are not received by the communication I/F 68, a negative determination is made, and the image management processing shifts to step ST300. In step ST300, in a case in which the patient specification information 90 and the hospitalization period information 96 are received by the communication I/F 68, a positive determination is made, and the image management processing shifts to step ST302.

In step ST302, the second search unit 62B searches the database 100 (see FIG. 6) to acquire the image information 102 (see FIG. 6) in accordance with the patient ID indicated by the patient specification information 90 received by the communication I/F 68 in step ST300 and the hospitalization period date 108B indicated by the hospitalization period information 96. After the processing of step ST302 is executed, the image management processing shifts to step ST304.

In step ST304, the fourth transmission/reception unit 62A transmits the image information 102 acquired in step ST302 to the medical support server 12. After the processing of step ST304 is executed, the image management processing ends.

Next, an example of a flow of the document management processing performed by the processor 72 of the document management server 18 will be described with reference to FIG. 16.

In the document management processing shown in FIG. 16, first, in step ST400, the fifth transmission/reception unit 72A determines whether or not the patient specification information 90 and the hospitalization period information 96 transmitted from the medical support server 12 by executing the processing of step ST112 included in the display screen generation processing shown in FIG. 13 are received by the communication I/F 78 (see FIG. 3). In step ST400, in a case in which the patient specification information 90 and the hospitalization period information 96 are not received by the communication I/F 78, a negative determination is made, and the document management processing shifts to step ST400. In step ST400, in a case in which the patient specification information 90 and the hospitalization period information 96 are received by the communication I/F 78, a positive determination is made, and the document management processing shifts to step ST402.

In step ST402, the third search unit 72B searches the database 104 (see FIG. 7) to acquire the document information 106 (see FIG. 7) in accordance with the patient ID indicated by the patient specification information 90 received by the communication I/F 78 in step ST400 and the hospitalization period date 108B indicated by the hospitalization period information 96. After the processing of step ST402 is executed, the document management processing shifts to step ST404.

In step ST404, the fifth transmission/reception unit 72A transmits the document information 106 acquired in step ST402 to the medical support server 12. After the processing of step ST404 is executed, the document management processing ends.

As described above, in the medical support system 10 according to the first embodiment, in a case in which the hospitalization period medical information 112 of the patient is displayed, the hospitalization period medical information 112 is disposed in a matrix in the first display region 110 defined by the horizontal axis B indicating the hospitalization period date 108B and the vertical axis A indicating the plurality of items 108A of the hospitalization period medical information 112. Therefore, with the present configuration, it is easy to grasp the hospitalization period medical information 112 of the patient as compared with a case in which all the medical information 120 from the past to the present is displayed.

Moreover, in the medical support system 10 according to the first embodiment, the number of days based on the hospitalization day 108C is displayed on at least one date included in the hospitalization period on the display screen 108. Therefore, with the present configuration, it is easy to grasp the hospitalization period medical information 112 based on the hospitalization day 108C as compared with a case in which the number of days since the hospitalization is not displayed.

Moreover, in the medical support system 10 according to the first embodiment, the surgery day 108D is displayed on the display screen 108 to be distinguishable from a date other than the surgery day 108D. Therefore, with the present configuration, it is easy to grasp the hospitalization period medical information 112 on the surgery day 108D as compared with a case in which the display of the surgery day 108D is the same as the display other than the surgery day 108D.

Moreover, in the medical support system 10 according to the first embodiment, on the display screen 108, the number of days based on the surgery day 108D is displayed on at least one date included in the hospitalization period. Therefore, with the present configuration, it is easy to grasp the hospitalization period medical information 112 based on the surgery day 108D as compared with a case in which the number of days since the surgery is not displayed.

Moreover, in the medical support system 10 according to the first embodiment, the icon 113 indicating that the medical information 120 is present is displayed in the cell 114 in the first display region 110, and the medical information 120 corresponding to the designated icon 113 is displayed in the medical information display region 116. Therefore, with the present configuration, it is easy to grasp the hospitalization period medical information 112 as compared with a case in which the medical information 120 is displayed in the first display region 110.

It should be noted that, in the first embodiment described above, the form example has been described in which all the hospitalization period dates 108B are displayed, but the technology of the present disclosure is not limited to this. For example, the date of the hospitalization period date 108B may be displayed every other day or every few days (for example, every three days), or may be only the date on which the hospitalization period medical information 112 is present among the hospitalization period dates 108B is displayed.

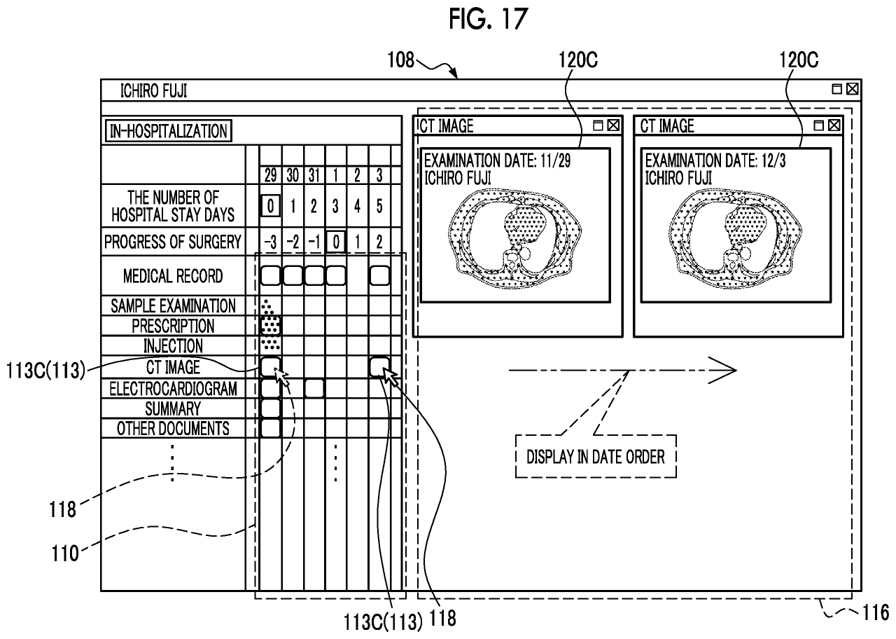

Moreover, in the first embodiment described above, the form example has been described in which a display order of the medical information 120 is not considered in the medical information display region 116, but the technology of the present disclosure is not limited to this. For example, as a modification example of one of the first embodiment, as shown in FIG. 17, the medical information 120 is displayed in the medical information display region 116 in a date order. That is, the medical information 120 is displayed in the medical information display region 116 in an order of the date (for example, an examination date or a creation date) indicated by the date information attached to the medical information 120. In an example shown in FIG. 17, a plurality of icons 113C are designated via the pointer 118. In this case, in the medical information display region 116, a plurality of CT images 120C indicated by the medical information 120 corresponding to the plurality of icons 113C are displayed from left to right in an order of an image on November 29th of the examination date, and the image on December 3rd of the examination date.

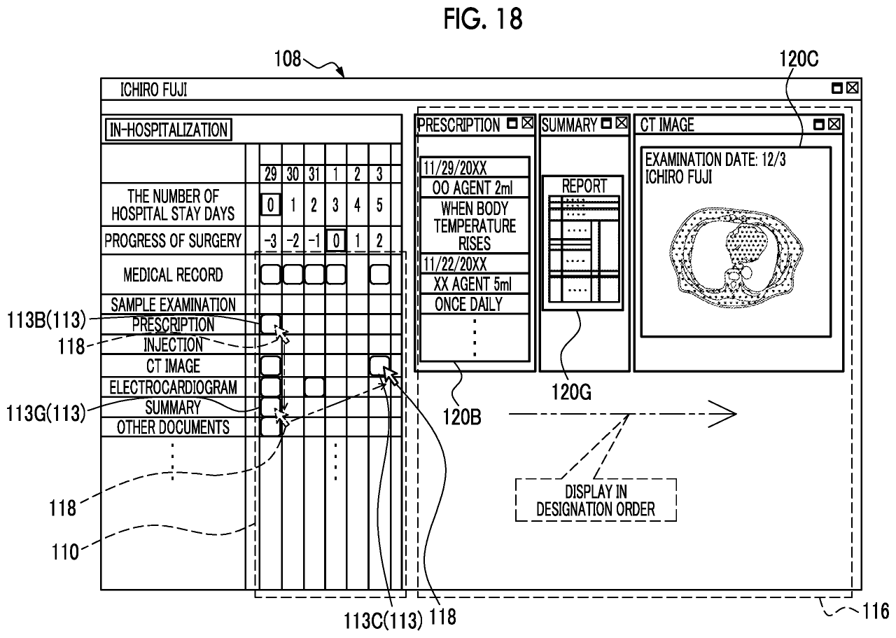

Moreover, as another modification example, as shown in FIG. 18, the medical information 120 is displayed in the medical information display region 116 in a designation order of the icons 113 in the first display region 110. In an example shown in FIG. 18, the plurality of icons 113B, 1136, and 113C are designated in an order via the pointer 118. In this case, in the medical information display region 116, the contents indicated by the medical information 120 corresponding to the plurality of icons 113 are displayed from left to right in a designation order. That is, the prescription 120B indicated by the medical information 120 corresponding to the icon 113B, the summary 120G indicated by the medical information 120 corresponding to the icon 113G and the CT image 120C indicated by the medical information 120 corresponding to the icon 113C are displayed in the medical information display region 116 from left to right.

Moreover, as another modification example, the medical information 120 may be displayed in the medical information display region 116 in an arrangement order of the plurality of items 108A in the direction of the vertical axis A. Moreover, whether the medical information 120 is displayed in any order of the designation order of the icons 113, the date order attached to the medical information 120, or the arrangement order of the plurality of items 108A in the direction of the vertical axis A may be switchable.

As described above, in the medical support system 10 according to the modification example of the first embodiment, in the medical information display region 116, the medical information 120 is displayed in any order of the designation order of the icons 113, the date order attached to the medical information 120, or the arrangement order of the plurality of items 108A in the direction of the vertical axis A. Therefore, with the present configuration, it is easy to grasp the medical information 120 in the hospitalization period as compared with a case in which the medical information 120 is always displayed in a fixed display order.

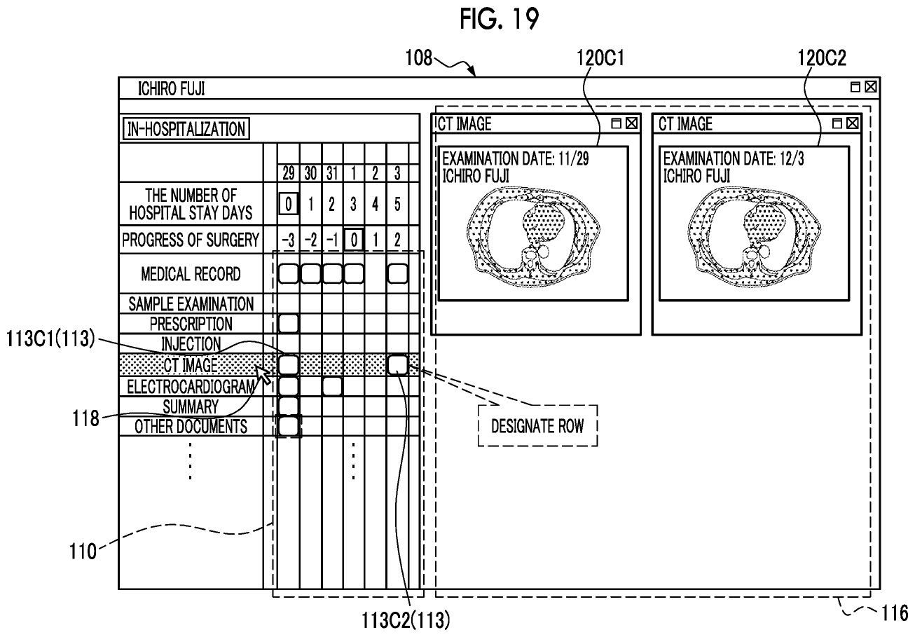

Moreover, in the first embodiment described above, the form example has been described in which the plurality of icons 113 are designated one by one, but the technology of the present disclosure is not limited to this. In the first display region 110, the icons 113 included in the row or column may be designated by designating the entire row or the entire column. In an example shown in FIG. 19, a row of the CT image is designated via the pointer 118. The row of the CT image includes icons 113C1 and 113C2. In the medical information display region 116, CT images 120C1 and 120C2 are displayed as an example of the contents indicated by the medical information 120 corresponding to the icons 113C1 and 113C2.

Moreover, in the first embodiment, the form example has been described in which the vertical axis A indicates the plurality of items 108A included in the hospitalization period medical information 112 and the horizontal axis B indicates the hospitalization period date 108B, but the technology of the present disclosure is not limited to this. For example, an aspect may be adopted in which the vertical axis A indicates the hospitalization period date 108B, and the horizontal axis B indicates the plurality of items 108A included in the hospitalization period medical information 112.

### Second Embodiment

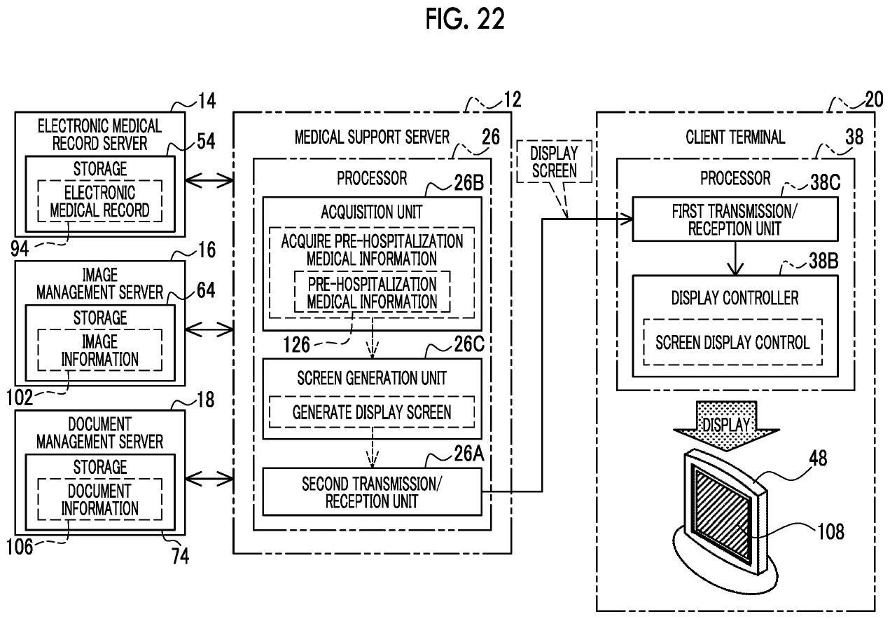

In the first embodiment, the case has been described in which the hospitalization period medical information 112 or the like is displayed on the display screen 108 for an in-hospitalization period, but the technology of the present disclosure is not limited to this. In the second embodiment, a case will be described in which the display screen 108 reflecting pre-hospitalization medical information 126 (see FIG. 22) and the like for a period before the patient is hospitalized (that is, a pre-hospitalization period) is displayed on the display 48.

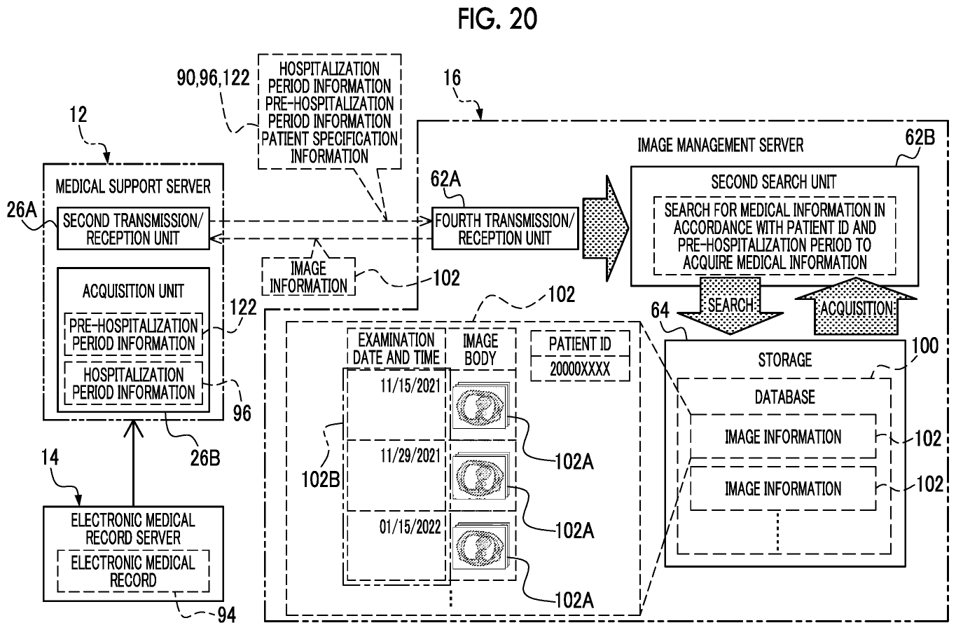

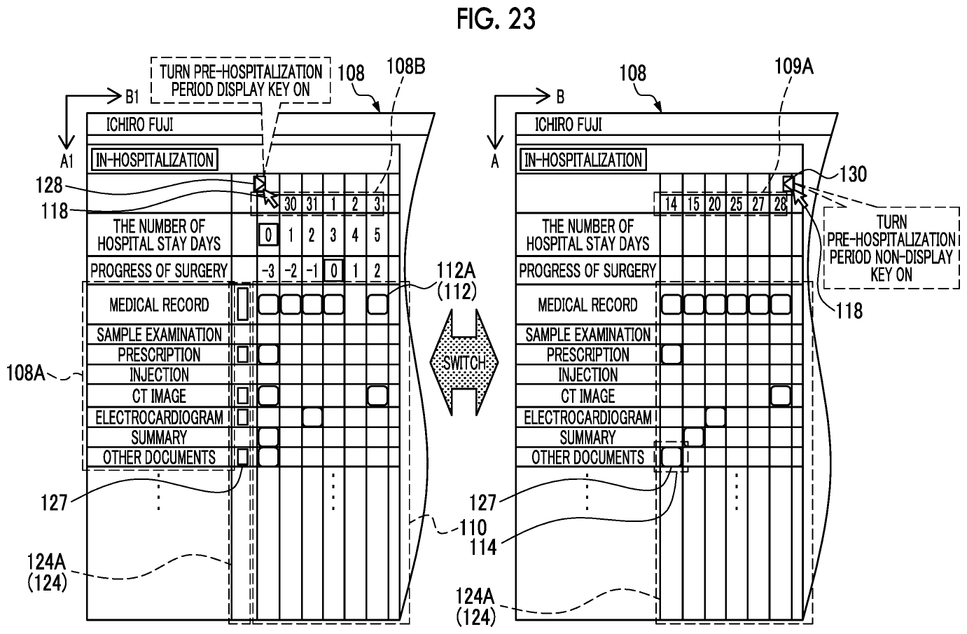

As shown in FIG. 20 as an example, the medical support server 12 acquires the electronic medical record 94 from the electronic medical record server 14. The acquisition unit 26B generates the hospitalization period information 96 indicating the hospitalization period date 108B based on the electronic medical record 94. Moreover, the acquisition unit 26B specifies a pre-hospitalization period date 109A (see FIG. 23) based on the electronic medical record 94. Here, the pre-hospitalization period date 109A refers to a date of the period before the patient is hospitalized. Moreover, the specification of the pre-hospitalization period date 109A refers to processing of calculating the period before the patient is hospitalized based on the date of the hospitalization day indicated by the electronic medical record 94. Stated another way, the pre-hospitalization period date 109A is determined based on a start day of the hospitalization period. Here, a predetermined period (for example, a period from the day before the hospitalization day to one month before the hospitalization day) is used as the period before hospitalization, but this is merely an example. For example, a range of the pre-hospitalization period received via the reception device 46 may be used to specify the pre-hospitalization period date 109A.

The acquisition unit 26B generates pre-hospitalization period information 122 indicating the pre-hospitalization period date 109A. The pre-hospitalization period date 109A is an example of a “non-hospitalization period date” according to the technology of the present disclosure. The second transmission/reception unit 26A of the medical support server 12 transmits the hospitalization period information 96, the pre-hospitalization period information 122, and the patient specification information 90 to the image management server 16. The fourth transmission/reception unit 62A of the image management server 16 receives the hospitalization period information 96, the pre-hospitalization period information 122, and the patient specification information 90.

The second search unit 62B searches the database 100 to acquire the image information 102 in accordance with the patient ID indicated by the patient specification information 90 received by the fourth transmission/reception unit 62A, the hospitalization period date 108B indicated by the hospitalization period information 96, and the pre-hospitalization period date 109A (see FIG. 23) indicated by the pre-hospitalization period information 122. In this case, for example, the second search unit 62B searches the database 100 to acquire the image information 102 in which the patient ID indicated by the patient specification information 90 and the image date information 102B that coincides with the hospitalization period date 108B indicated by the hospitalization period information 96 are included. Moreover, the second search unit 62B searches the database 100 to acquire the image information 102 in which the patient ID indicated by the patient specification information 90 and the image date information 102B that coincides with the pre-hospitalization period date 109A indicated by the pre-hospitalization period information 122 are included.

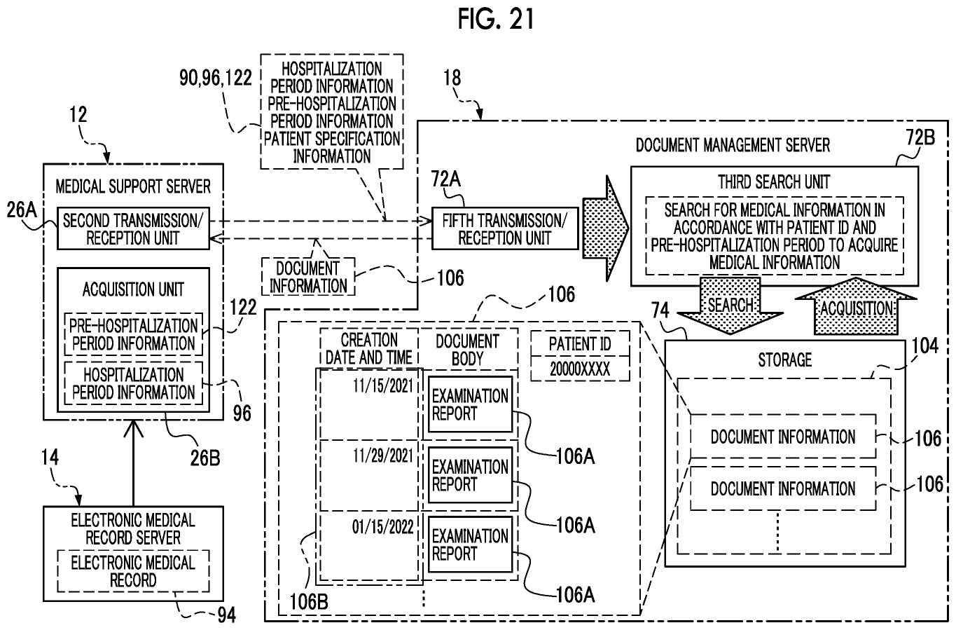

As shown in FIG. 21 as an example, the second transmission/reception unit 26A of the medical support server 12 transmits the hospitalization period information 96, the pre-hospitalization period information 122, and the patient specification information 90 to the document management server 18. The fifth transmission/reception unit 72A of the document management server 18 receives the hospitalization period information 96, the pre-hospitalization period information 122, and the patient specification information 90.

The third search unit 72B searches the database 104 to acquire the document information 106 in accordance with the patient ID indicated by the patient specification information 90 received by the fifth transmission/reception unit 72A, the hospitalization period date 108B indicated by the hospitalization period information 96, and the pre-hospitalization period date 109A (see FIG. 23) indicated by the pre-hospitalization period information 122. In this case, for example, the third search unit 72B searches the database 104 to acquire the document information 106 in which the patient ID indicated by the patient specification information 90 and the document date information 106B that coincides with the hospitalization period date 108B indicated by the hospitalization period information 96 are included. Moreover, the third search unit 72B searches the database 104 to acquire the document information 106 in which the patient ID indicated by the patient specification information 90 and the document date information 106B that coincides with the pre-hospitalization period date 109A indicated by the pre-hospitalization period information 122 are included.

In the document management server 18, the fifth transmission/reception unit 72A transmits the document information 106 acquired by the third search unit 72B to the medical support server 12. In the medical support server 12, the second transmission/reception unit 26A receives the document information 106 transmitted from the document management server 18.

The plurality of items 108A (see FIG. 23) included in the electronic medical record 94, the image information 102, and the document information 106 acquired by the acquisition unit 26B are associated with the pre-hospitalization period date 109A (see FIG. 23). That is, as shown in FIG. 23 as an example, the acquisition unit 26B acquires the pre-hospitalization medical information 126 including the plurality of items 108A associated with the pre-hospitalization period date 109A from the electronic medical record server 14, the image management server 16, and the document management server 18. It should be noted that the plurality of items 108A are an example of a “plurality of second items” according to the technology of the present disclosure, and the pre-hospitalization medical information 126 is an example of “non-hospitalization period medical information” according to the technology of the present disclosure.

The screen generation unit 26C generates the display screen 108 based on the pre-hospitalization medical information 126 acquired by the acquisition unit 26B. That is, the screen generation unit 26C generates the display screen 108 on which the pre-hospitalization medical information 126 is disposed in a matrix in a region in which the pre-hospitalization period date 109A is on a horizontal axis B1 (see FIG. 23), and the plurality of items 108A (see FIG. 23) included in the electronic medical record 94, the image information 102, and the document information 106 are on a vertical axis A1 (see FIG. 23).

The second transmission/reception unit 26A transmits the information indicating the display screen 108 generated by the screen generation unit 26C to the client terminal 20.

As an example, as shown in FIG. 23, the hospitalization period medical information 112 is disposed in a matrix in the first display region 110 on the display screen 108. Further, a second display region 124 is displayed on the display screen 108. In an example shown in FIG. 23, a pre-hospitalization display region 124A is displayed as the second display region 124 on the display screen 108. The pre-hospitalization display region 124A is a region defined by the vertical axis A1 and the horizontal axis B1. The vertical axis A1 refers to an axis indicating the plurality of items 108A included in the pre-hospitalization medical information 126. The horizontal axis B1 refers to an axis indicating the pre-hospitalization period date 109A. In the example shown in FIG. 23, an axis extending in the same direction as the vertical axis A is shown as an example of the vertical axis A1, and an axis extending in the same direction as the horizontal axis B is shown as an example of the horizontal axis B1. It should be noted that the second display region 124 is an example of a “second display region” according to the technology of the present disclosure, and the pre-hospitalization display region 124A is an example of a “pre-hospitalization display region” according to the technology of the present disclosure. Moreover, the vertical axis A1 is an example of a “fourth axis” according to the technology of the present disclosure, and the horizontal axis B1 is an example of a “third axis” according to the technology of the present disclosure.

The pre-hospitalization medical information 126 is disposed in a matrix in the pre-hospitalization display region 124A. Here, the fact that the pre-hospitalization medical information 126 is disposed in a matrix includes an aspect in which display indicating the presence, a state, or a type of the medical information 120 corresponding to the pre-hospitalization medical information 126 is disposed, in addition to an aspect in which the pre-hospitalization medical information 126 itself is disposed. In the example shown in FIG. 23, quadrangular icons 127 indicating the presence of the medical information 120 are disposed in a matrix in the pre-hospitalization display region 124A. In the example shown in FIG. 23, the icon 127 is displayed in the cell 114 at a position at which a row of the corresponding item among the plurality of items 108A intersects with a column of the corresponding date among the pre-hospitalization period dates 109A. That is, the icon 127 indicates the presence of the medical information 120 corresponding to the cell 114.

As an example, as shown on the left side of the front view of FIG. 23, in the pre-hospitalization display region 124A, a date for a first number which is smaller than the number of dates included in the pre-hospitalization period date 109A is displayed as the pre-hospitalization period date 109A on the horizontal axis B1. That is, in the pre-hospitalization display region 124A, the horizontal axis B1 indicating the pre-hospitalization period date 109A is displayed in a reduced state. In the example shown in FIG. 23, as the horizontal axis B1 of the pre-hospitalization display region 124A, a date for one day smaller than six days which are the number of dates included in the pre-hospitalization period date 109A is displayed.

In the pre-hospitalization display region 124A, the number of dates indicated by the horizontal axis B1 can be switched between the first number and a second number larger than the first number. The date for one day is displayed as the first number in the example shown on the left side of the front view of FIG. 23, and the dates for six days are displayed as the second number in the example shown on the right side of the front view of FIG. 23. That is, in the pre-hospitalization display region 124A, the horizontal axis B1 indicating the pre-hospitalization period date 109A can be switched between the reduced display state and the enlarged display state. In the example shown in FIG. 23, a pre-hospitalization period display key 128 is displayed on the display screen 108 in a state in which the pre-hospitalization period date 109A is reduced and displayed in one column. Here, in a case in which the pre-hospitalization period display key 128 is turned on via the pointer 118, the horizontal axis B1 indicating the pre-hospitalization period date 109A is enlarged and displayed as shown on the right side of the front view of FIG. 23.

In the example shown in FIG. 23, in a state in which the horizontal axis B1 indicating the pre-hospitalization period date 109A is enlarged and displayed, the horizontal axis B1 indicates only the date on which the pre-hospitalization medical information 126 is disposed among the pre-hospitalization period dates 109A. Stated another way, in a state in which the horizontal axis B1 indicating the pre-hospitalization period date 109A is enlarged and displayed, not all the pre-hospitalization period dates 109A are displayed, but only the date on which the pre-hospitalization medical information 126 corresponding to the date is present is displayed. It should be noted that, in the example shown in FIG. 23, the example has been described in which the horizontal axis B1 indicates only the date on which the pre-hospitalization medical information 126 is disposed among the non-hospitalization period dates, but this is merely an example. For example, in a state in which the horizontal axis B1 is enlarged and displayed, all dates of the pre-hospitalization period dates 109A may be displayed, or only the date for a predetermined number of days (for example, one week) among the pre-hospitalization period dates 109A may be displayed. Moreover, a period from the last hospital discharge day to the current hospitalization day may be displayed as the date of the pre-hospitalization period date 109A.

Moreover, the date of the pre-hospitalization period date 109A may be displayed at intervals of every other day or every few days (for example, every three days). Further, the interval at which the pre-hospitalization period date 109A is displayed may be a smaller number of days than the interval at which the hospitalization period date 108B is displayed. That is, in a case in which the date of the hospitalization period date 108B is displayed at intervals of Y days under a condition that a magnitude relationship of “Y<X” is established, the date of the pre-hospitalization period date 109A may be displayed at intervals of X days.

In the example shown on the right side of the front view of FIG. 23, a pre-hospitalization period non-display key 130 is displayed on the display screen 108 in a state in which the horizontal axis B1 indicating the pre-hospitalization period date 109A is enlarged and displayed. Here, in a case in which the pre-hospitalization period non-display key 130 is turned on via the pointer 118, the horizontal axis B1 indicating the pre-hospitalization period date 109A is reduced and displayed as shown on the left side of the front view of FIG. 23. As described above, in the pre-hospitalization display region 124A, the horizontal axis B1 indicating the pre-hospitalization period date 109A can be switched between the reduced display state and the enlarged display state.

As described above, in the medical support system 10 according to the second embodiment, in a case in which the pre-hospitalization medical information 126 of the patient is displayed, the pre-hospitalization medical information 126 is disposed in a matrix in the pre-hospitalization display region 124A defined by the horizontal axis B1 indicating the pre-hospitalization period date 109A and the vertical axis A1 indicating the items 108A of the pre-hospitalization medical information 126. Therefore, with the present configuration, it is easy to grasp the pre-hospitalization medical information 126 of the patient as compared with a case in which all the medical information 120 from the past to the present is displayed. Moreover, the display screen 108 includes the pre-hospitalization display region 124A in which the pre-hospitalization medical information 126 is displayed, in addition to the first display region 110 in which the hospitalization period medical information 112 is disposed. Therefore, with the present configuration, the pre-hospitalization medical information 126 related to the hospitalization period medical information 112 can be confirmed as compared with a case in which the pre-hospitalization medical information 126 is not displayed.

Moreover, in the medical support system 10 according to the second embodiment, the pre-hospitalization period date 109A is determined based on the start day of the hospitalization period. Therefore, with the present configuration, it is easy to grasp the pre-hospitalization medical information 126 as compared with a case in which all the pre-hospitalization medical information 126 is displayed. Moreover, the display screen 108 includes the pre-hospitalization display region 124A in which the pre-hospitalization medical information 126 based on the start day of the specific hospitalization period is displayed, in addition to the first display region 110 in which the hospitalization period medical information 112 of a specific hospitalization period is disposed. Therefore, with the present configuration, it is easy to grasp the pre-hospitalization medical information 126 related to the hospitalization period medical information 112 as compared with a case in which the pre-hospitalization medical information 126 of a period not related to the specific hospitalization period is displayed.

Moreover, in the medical support system 10 according to the second embodiment, the horizontal axis B1 of the pre-hospitalization display region 124A indicates the date for the first number, which is a number smaller than the number of dates included in the pre-hospitalization period date 109A. Therefore, with the present configuration, it is easy to grasp the pre-hospitalization medical information 126 as compared with a case in which all the dates included in the pre-hospitalization period date 109A are displayed. Stated another way, the number of dates displayed in the first display region 110 in which the hospitalization period medical information 112 is disposed is larger than the first number which is the number of dates displayed in the pre-hospitalization display region 124A in which the pre-hospitalization medical information 126 is displayed. Therefore, with the present configuration, as compared with a case in which the number of dates displayed in the first display region 110 in which the hospitalization period medical information 112 is disposed is smaller than the first number which is the number of dates displayed in the pre-hospitalization display region 124A in which the pre-hospitalization medical information 126 is displayed, it is possible to secure the first display region 110 of the hospitalization period medical information 112, and it is easier to grasp the hospitalization period medical information 112.

Moreover, in the medical support system 10 according to the second embodiment, the number of dates indicated by the horizontal axis B1 of the pre-hospitalization display region 124A can be switched between the first number and the second number larger than the first number. Therefore, with the present configuration, it is easy to grasp the pre-hospitalization medical information 126 as compared with a case in which the number of dates indicated by the horizontal axis B1 is always the same. Stated another way, with respect to the number of dates displayed in the first display region 110 in which the hospitalization period medical information 112 is disposed, the number of dates displayed in the pre-hospitalization display region 124A in which the pre-hospitalization medical information 126 is displayed is variable between the first number and the second number. Therefore, with the present configuration, as compared with a case in which the number of dates displayed in the pre-hospitalization display region 124A in which the pre-hospitalization medical information 126 is displayed cannot be changed between the first number and the second number, it is possible to switch the ease of grasping the pre-hospitalization medical information 126 with respect to the hospitalization period medical information 112 in response to an instruction of the user or the like.

Moreover, in the medical support system 10 according to the second embodiment, the horizontal axis B1 indicating the pre-hospitalization period date 109A indicates the date on which the pre-hospitalization medical information 126 is disposed. Therefore, with the present configuration, it is easy to grasp the pre-hospitalization medical information 126 as compared with a case in which the number of dates indicated by the horizontal axis B1 is always fixed. For example, since the date on which the pre-hospitalization medical information 126 is not disposed is not displayed and only the date on which the pre-hospitalization medical information 126 is disposed is displayed, it is easy to grasp the pre-hospitalization medical information 126 as compared with a case in which the number of dates indicated by the horizontal axis B1 is always fixed.

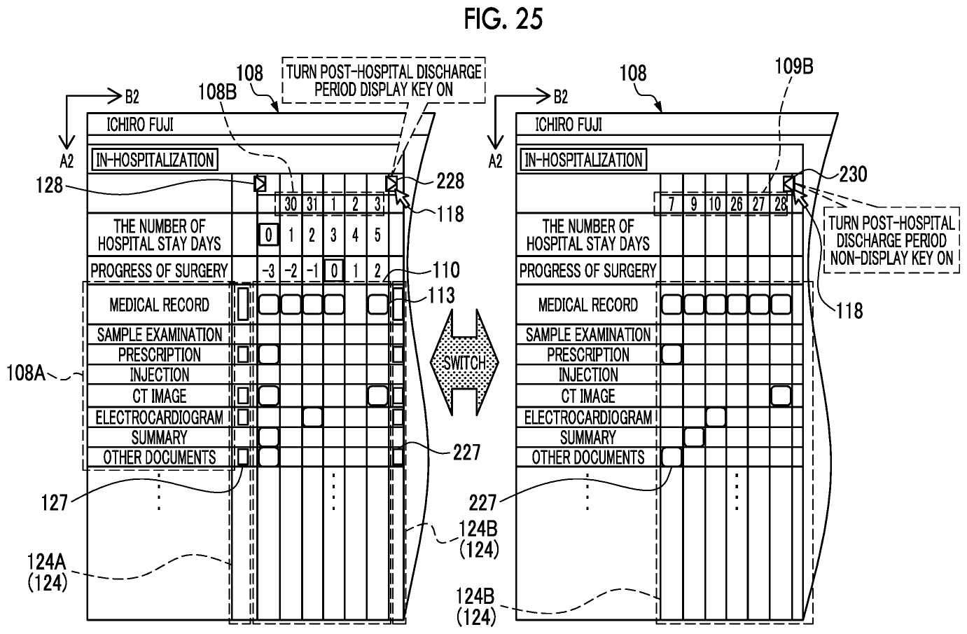

It should be noted that, in the second embodiment described above, the form example has been described in which the pre-hospitalization medical information 126 is displayed in the pre-hospitalization display region 124A, but the technology of the present disclosure is not limited to this. In the present modification example, post-hospital discharge medical information 226 and the like (see FIG. 25) are also displayed on the display screen 108 for a period after the patient is discharged from the hospital (that is, a post-hospital discharge period).

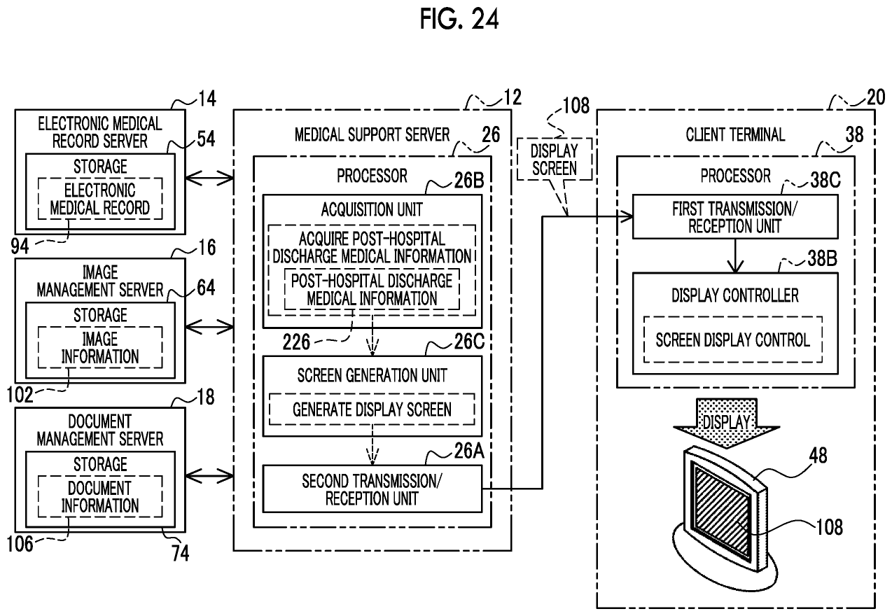

As shown in FIG. 24 as an example, the medical support server 12 acquires the electronic medical record 94 from the electronic medical record server 14. The acquisition unit 26B generates the hospitalization period information 96 indicating the hospitalization period date 108B (see FIG. 23) based on the electronic medical record 94. Moreover, the acquisition unit 26B specifies a post-hospital discharge period date 109B (see FIG. 25) based on the electronic medical record 94. Here, the post-hospital discharge period date 109B refers to a date after the patient is discharged from the hospital. Moreover, the specification of the post-hospital discharge period date 109B refers to processing of calculating a period after the patient is discharged from the hospital based on the date of the hospital discharge day indicated by the electronic medical record 94. The post-hospital discharge period date 109B is determined based on the end day of the hospitalization period. For example, the post-hospital discharge period date 109B may be one month from the end day of the hospitalization period. The acquisition unit 26B generates post-hospital discharge period information 222 indicating the post-hospital discharge period date 109B. The post-hospital discharge period date 109B is an example of a “non-hospitalization period date” according to the technology of the present disclosure.

The acquisition unit 26B acquires the image information 102 in accordance with the post-hospital discharge period date 109B from the image management server 16. Specifically, the acquisition unit 26B acquires the image information 102 in which the image date information 102B (see FIG. 7) indicating the post-hospital discharge period date 109B from the image management server 16 is included. Moreover, the acquisition unit 26B acquires the document information 106 in accordance with the post-hospital discharge period date 109B from the document management server 18. Specifically, the acquisition unit 26B acquires the document information 106 in which the document date information 106B (see FIG. 8) indicating the post-hospital discharge period date 109B from the document management server 18 is included.

It should be noted that, in the specification of the post-hospital discharge period date 109B by the acquisition unit 26B, in a case in which the hospital discharge day (that is, the end day of the hospital discharge period) is not present, the acquisition unit 26B does not acquire the image information 102 in accordance with the post-hospital discharge period date 109B from the image management server 16. Moreover, in the specification of the post-hospital discharge period date 109B by the acquisition unit 26B, in a case in which the hospital discharge day is not present, the acquisition unit 26B does not acquire the document information 106 in accordance with the post-hospital discharge period date 109B from the document management server 18. Therefore, a post-hospital discharge display region 124B (see FIG. 25) is not generated by the screen generation unit 26C described below. Stated another way, the post-hospital discharge display region 124B is displayed on the display screen 108 in a case in which the end day of the hospitalization period is present.

As described above, the plurality of items 108A (see FIG. 25) are included in the electronic medical record 94, the image information 102, and the document information 106 acquired by the acquisition unit 26B. Moreover, the post-hospital discharge period date 109B (see FIG. 24) is associated with the electronic medical record 94, the image information 102, and the document information 106 acquired by the acquisition unit 26B. That is, the acquisition unit 26B acquires the post-hospital discharge medical information 226 including the plurality of items 108A (see FIG. 25) associated with the post-hospital discharge period date 109B (see FIG. 24). The post-hospital discharge medical information 226 is an example of “non-hospitalization period medical information” according to the technology of the present disclosure.

The screen generation unit 26C generates the display screen 108 based on the post-hospital discharge medical information 226 acquired by the acquisition unit 26B. That is, the screen generation unit 26C generates the display screen 108 based on the electronic medical record 94, the image information 102, and the document information 106 in accordance with the post-hospital discharge period date 109B. That is, the screen generation unit 26C generates the display screen 108 on which the post-hospital discharge medical information 226 is disposed in a matrix in a region in which the post-hospital discharge period date 109B is on a horizontal axis B2 (see FIG. 25), and the plurality of items 108A (see FIG. 25) included in the electronic medical record 94, the image information 102, and the document information 106 are on a vertical axis A2 (see FIG. 25).

As shown in FIG. 25 as an example, the post-hospital discharge display region 124B is displayed as the second display region 124 on the display screen 108. The post-hospital discharge display region 124B is a region defined by the vertical axis A2 and the horizontal axis B2. The vertical axis A2 refers to an axis indicating the plurality of items 108A included in the post-hospital discharge medical information 226. The horizontal axis B2 indicates an axis indicating the post-hospital discharge period date 109B. In an example shown in FIG. 25, an axis extending in the same direction as the vertical axis A is shown as an example of the vertical axis A2, and an axis extending in the same direction as the horizontal axis B is shown as an example of the horizontal axis B2. The post-hospital discharge display region 124B is an example of a “post-hospital discharge display region” according to the technology of the present disclosure. Moreover, the vertical axis A2 is an example of a “fourth axis” according to the technology of the present disclosure, and the horizontal axis B2 is an example of a “third axis” according to the technology of the present disclosure.

The post-hospital discharge medical information 226 is disposed in a matrix in the post-hospital discharge display region 124B. Here, the fact that the post-hospital discharge medical information 226 is disposed in a matrix includes an aspect in which display indicating the presence, a state, or a type of the medical information 120 corresponding to the post-hospital discharge medical information 226 is disposed, in addition to an aspect in which the post-hospital discharge medical information 226 itself is disposed. In the example shown in FIG. 25, quadrangular icons 227 indicating the presence of the post-hospital discharge medical information 226 are disposed in a matrix in the post-hospital discharge display region 124B. In the example shown in FIG. 25, the icon 227 is displayed in the cell 114 at a position at which a row of the corresponding item 108A among the plurality of items 108A intersects with a column of the corresponding date among the post-hospital discharge period dates 109B. That is, the icon 227 indicates the presence of the medical information 120 corresponding to the cell 114.

As an example, as shown on the left side of the front view of FIG. 25, in the post-hospital discharge display region 124B, a date for the first number which is smaller than the number of dates included in the post-hospital discharge period date 109B is displayed as the post-hospital discharge period date 109B on the horizontal axis B2. Moreover, in the post-hospital discharge display region 124B, the number of dates indicated by the horizontal axis B2 can be switched between the first number and the second number larger than the first number.

In a state in which the post-hospital discharge period date 109B is reduced and displayed (that is, displayed by the first number), in a case in which a post-hospital discharge period display key 228 is turned on via the pointer 118, as shown on the right side of the front view of FIG. 25, the horizontal axis B2 indicating the post-hospital discharge period date 109B is enlarged and displayed (that is, displayed by the second number). In the example shown on the right side of the front view of FIG. 25, in a case in which a post-hospital discharge period non-display key 230 is turned on via the pointer 118, as shown on the left side of the front view of FIG. 25, the horizontal axis B2 indicating the post-hospital discharge period date 109B is reduced and displayed. As described above, in the post-hospital discharge display region 124B, the horizontal axis B2 indicating the post-hospital discharge period date 109B can be switched between the reduced display state and the enlarged display state.

In the example shown in FIG. 25, in a case in which a plurality of dates are included in the post-hospital discharge period date 109B, the horizontal axis B2 indicates only the date on which the post-hospital discharge medical information 226 is disposed among the post-hospital discharge period dates 109B. Stated another way, in a state in which the horizontal axis B2 indicating the post-hospital discharge period date 109B is enlarged and displayed, not all the post-hospital discharge period dates 109B are displayed, but only the date on which the post-hospital discharge medical information 226 is present is displayed. It should be noted that, in the example shown in FIG. 25, the example has been described in which the horizontal axis B2 indicates only the date on which the post-hospital discharge medical information 226 is disposed among the post-hospital discharge period dates 109B, but this is merely an example. For example, in a state in which the horizontal axis B2 is enlarged and displayed, in a case in which a plurality of dates are included in the post-hospital discharge period date 109B, all the dates of the post-hospital discharge period date 109B may be displayed, or only the date for a predetermined number of days (for example, one week) among the post-hospital discharge period dates 109B may be displayed. Moreover, a period from the current hospital discharge day to the next hospitalization day may be displayed as the date of the pre-hospitalization period date 109A. It should be noted that the post-hospital discharge period date 109B may be a single date.

Moreover, the date of the post-hospital discharge period date 109B may be displayed at intervals of every other day or every few days (for example, every three days). Further, the interval at which the post-hospital discharge period date 109B is displayed may be a smaller number of days than the interval at which the hospitalization period date 108B is displayed. That is, in a case in which the date of the hospitalization period date 108B is displayed at intervals of Y days under a condition that a magnitude relationship of “Y<X” is established, the date of the post-hospital discharge period date 109B may be displayed at intervals of X days.

As described above, in the medical support system 10 according to the modification example of the second embodiment, in a case in which the post-hospital discharge medical information 226 of the patient is displayed, the post-hospital discharge medical information 226 is disposed in a matrix in the post-hospital discharge display region 124B defined by the horizontal axis B2 indicating the post-hospital discharge period date 109B and the vertical axis A2 indicating the items 108A of the post-hospital discharge medical information 226. Therefore, with the present configuration, it is easy to grasp the post-hospital discharge medical information 226 of the patient as compared with a case in which all the medical information 120 from the past to the present is displayed. Moreover, the display screen 108 includes the post-hospital discharge display region 124B in which the post-hospital discharge medical information 226 is displayed, in addition to the first display region 110 in which the hospitalization period medical information 112 is disposed. Therefore, with the present configuration, the post-hospital discharge medical information 226 related to the hospitalization period medical information 112 can be confirmed as compared with a case in which the post-hospital discharge medical information 226 is not displayed.

Moreover, in the medical support system 10 according to a modification example of the second embodiment, the post-hospital discharge period date 109B is determined based on the start day of the hospitalization period. Therefore, with the present configuration, it is easy to grasp the post-hospital discharge medical information 226 as compared with a case in which all the post-hospital discharge medical information 226 is displayed. Moreover, the display screen 108 includes the post-hospital discharge display region 124B in which the post-hospital discharge medical information 226 based on the start day of the specific hospitalization period is displayed, in addition to the first display region 110 in which the hospitalization period medical information 112 of a specific hospitalization period is disposed. Therefore, with the present configuration, it is easy to grasp the post-hospital discharge medical information 226 related to the hospitalization period medical information 112 as compared with a case in which the post-hospital discharge medical information 226 of a period not related to the specific hospitalization period is displayed.

Moreover, in the medical support system 10 according to the modification example of the second embodiment, the horizontal axis B2 of the post-hospital discharge display region 124B indicates the date for the first number, which is a number smaller than the number of dates included in the post-hospital discharge period date 109B. Therefore, with the present configuration, it is easy to grasp the post-hospital discharge medical information 226 as compared with a case in which all the dates included in the post-hospital discharge period date 109B are displayed. Stated another way, the number of dates displayed in the first display region 110 in which the hospitalization period medical information 112 is disposed is larger than the first number which is the number of dates displayed in the post-hospital discharge display region 124B in which the post-hospital discharge medical information 226 is displayed. Therefore, with the present configuration, as compared with a case in which the number of dates displayed in the first display region 110 in which the hospitalization period medical information 112 is disposed is smaller than the first number which is the number of dates displayed in the post-hospital discharge display region 124B in which the post-hospital discharge medical information 226 is displayed, it is possible to secure the first display region 110 of the hospitalization period medical information 112, and it is easier to grasp the hospitalization period medical information 112.

Moreover, in the medical support system 10 according to the modification example of the second embodiment, the number of dates indicated by the horizontal axis B2 of the post-hospital discharge display region 124B can be switched between the first number and the second number larger than the first number. Therefore, with the present configuration, it is easy to grasp the post-hospital discharge medical information 226 as compared with a case in which the number of dates indicated by the horizontal axis B2 is always the same. Stated another way, with respect to the number of dates displayed in the first display region 110 in which the hospitalization period medical information 112 is disposed, the number of dates displayed in the post-hospital discharge display region 124B in which the post-hospital discharge medical information 226 is displayed is variable between the first number and the second number. Therefore, with the present configuration, as compared with a case in which the number of dates displayed in the post-hospital discharge display region 124B in which the post-hospital discharge medical information 226 is displayed cannot be changed between the first number and the second number, it is possible to switch the ease of grasping the post-hospital discharge medical information 226 with respect to the hospitalization period medical information 112 in response to an instruction of the user or the like.

Moreover, in the medical support system 10 according to the modification example of the second embodiment, the horizontal axis B2 indicating the post-hospital discharge period date 109B indicates the date on which the post-hospital discharge medical information 226 is disposed. Therefore, with the present configuration, it is easy to grasp the post-hospital discharge medical information 226 as compared with a case in which the number of dates indicated by the horizontal axis B2 is always fixed. For example, since the date on which the post-hospital discharge medical information 226 is not disposed is not displayed and only the date on which the post-hospital discharge medical information 226 is disposed is displayed, it is easy to grasp the post-hospital discharge medical information 226 as compared with a case in which the number of dates indicated by the horizontal axis B2 is always fixed.

Moreover, in the medical support system 10 according to the modification example of the second embodiment, the second display region 124 includes the pre-hospitalization display region 124A and the post-hospital discharge display region 124B, and the post-hospital discharge display region 124B is displayed in a case in which the end day of the hospitalization period is present. Therefore, with the present configuration, it is easy to grasp the pre-hospitalization medical information 126 and the post-hospital discharge medical information 226 as compared with a case in which the post-hospital discharge display region 124B is always displayed. Moreover, the display screen 108 includes the post-hospital discharge display region 124B in which the post-hospital discharge medical information 226 is displayed, in addition to the first display region 110 in which the hospitalization period medical information 112 is disposed. Therefore, with the present configuration, it is easy to grasp the hospitalization period medical information 112 as compared with a case in which the post-hospital discharge display region 124B is always displayed.

Moreover, in the second embodiment, the form example has been described in which the vertical axis A1 indicates the plurality of items 108A included in the pre-hospitalization medical information 126 and the horizontal axis B1 indicates the pre-hospitalization period date 109A, but the technology of the present disclosure is not limited to this. For example, an aspect may be adopted in which the vertical axis A1 indicates the pre-hospitalization period date 109A, and the horizontal axis B1 indicates the plurality of items 108A included in the pre-hospitalization medical information 126. Further, in the second embodiment, the form example has been described in which the vertical axis A2 indicates the plurality of items 108A included in the post-hospital discharge medical information 226, and the horizontal axis B2 indicates the post-hospital discharge period date 109B, but the technology of the present disclosure is not limited to this. For example, an aspect may be adopted in which the vertical axis A2 indicates the post-hospital discharge period date 109B, and the horizontal axis B2 indicates the plurality of items 108A included in the post-hospital discharge medical information 226.

### Third Embodiment

In the first and second embodiments, the case has been described in which the width (that is, a range in a direction of the horizontal axis B) of the first display region 110 is fixed in the display screen 108, but the technology of the present disclosure is not limited to this. In the third embodiment, a case will be described in which the width of the first display region 110 is changed in accordance with the number of dates included in the hospitalization period date 108B in a case in which a plurality of dates are included in the hospitalization period date 108B.

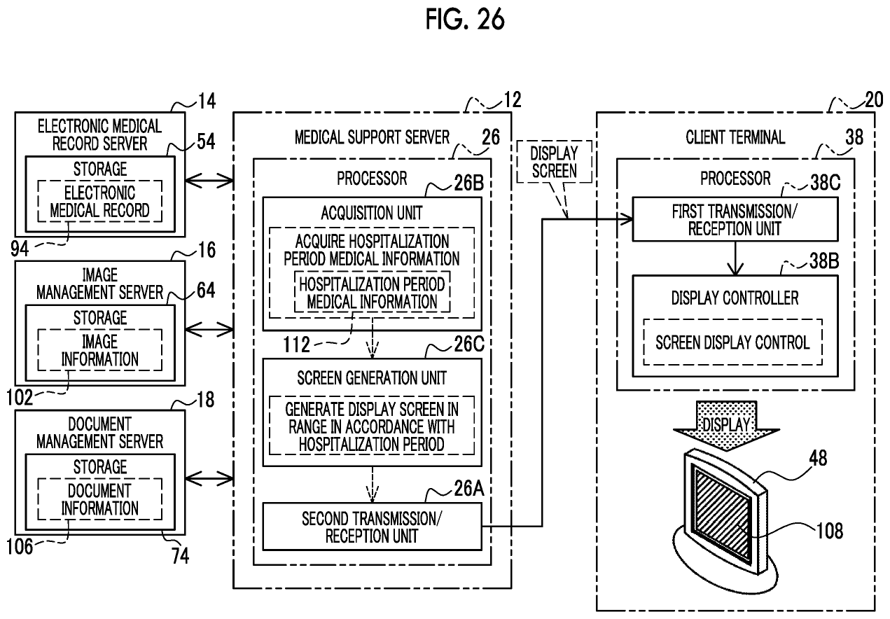

As shown in FIG. 26 as an example, the acquisition unit 26B acquires the image information 102 in accordance with the hospitalization period date 108B from the image management server 16. Specifically, the acquisition unit 26B acquires the image information 102 in which the image date information 102B (see FIG. 7) indicating the hospitalization period date 108B is included from the image management server 16. Moreover, the acquisition unit 26B acquires the document information 106 in accordance with the hospitalization period date 108B from the document management server 18. Specifically, the acquisition unit 26B acquires the document information 106 in which the document date information 106B (see FIG. 8) indicating the hospitalization period date 108B is included from the document management server 18.

The screen generation unit 26C generates the display screen 108 based on the electronic medical record 94, the image information 102, and the document information 106 in accordance with the hospitalization period date 108B. That is, the screen generation unit 26C generates the display screen 108 on which the hospitalization period medical information 112 is disposed in a matrix in the region in which the hospitalization period date 108B is on the horizontal axis B (see FIG. 10), and the plurality of items 108A (see FIG. 10) included in the electronic medical record 94, the image information 102, and the document information 106 are on the vertical axis A (see FIG. 10).

Here, the screen generation unit 26C adjusts the width (that is, the range in the direction of the horizontal axis B) of the first display region 110 in accordance with the hospitalization period. For example, the screen generation unit 26C derives the width of the first display region 110 by using width derivation information (not shown). The width derivation information refers to information in which the number of dates is associated with the width of the first display region 110. Examples of the width derivation information include a table in which the number of dates is used as input and the width of the first display region 110 is used as output, or an arithmetic expression in which the number of dates is used as an independent variable and the width of the first display region 110 is used as a dependent variable. The screen generation unit 26C generates the display screen 108 based on the calculated width of the first display region 110.

The second transmission/reception unit 26A transmits the information indicating the display screen 108 generated by the screen generation unit 26C to the client terminal 20. In the client terminal 20, the first transmission/reception unit 38C receives the information indicating the display screen 108. The display screen 108 is displayed on the display 48 under the control of the display controller 38A.

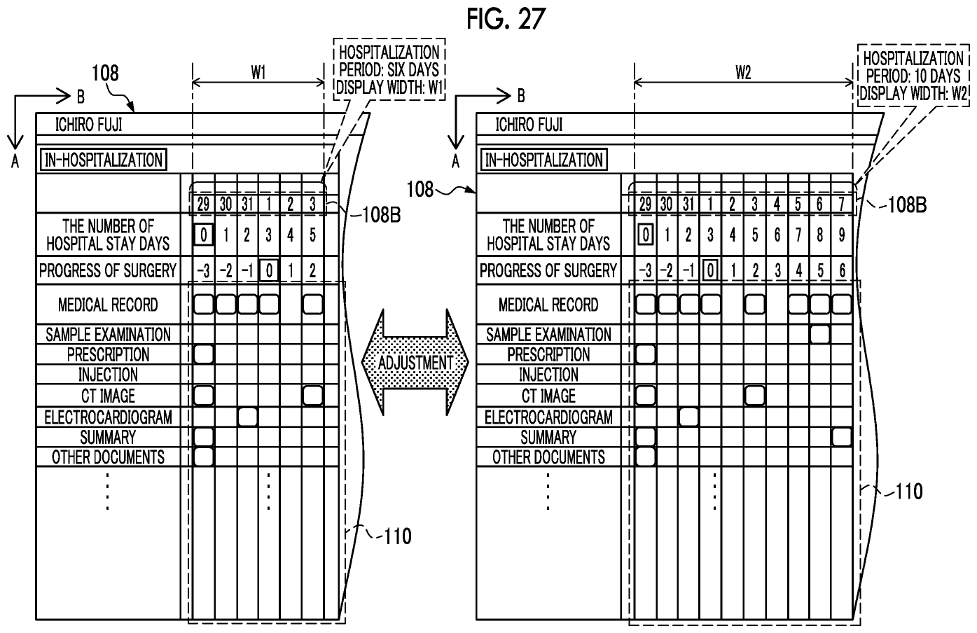

On the display screen 108, the width of the first display region 110 is adjusted such that the width is increased as the number of hospitalization period dates 108B is increased and the width is decreased as the number of hospitalization period dates 108B is decreased. In the example shown on the left side of the front view of FIG. 27, a case is shown in which the hospitalization period is six days, and the width of the first display region 110 is set to W1. On the other hand, as shown on the right side of the front view of FIG. 27, in a case in which the hospitalization period is 10 days, the width of the first display region 110 is set to W2. Here, the width W2 is a value larger than the width W1. As described above, on the display screen 108, the width of the first display region 110 is determined in accordance with the hospitalization period. The widths W1 and W2 are examples of a “range in a direction of the first axis” according to the technology of the present disclosure.

As described above, in the medical support system 10 according to the third embodiment, the width of the first display region 110 is determined in accordance with the hospitalization period. Therefore, with the present configuration, it is easy to grasp the hospitalization period medical information 112 as compared with a case in which the width of the first display region 110 is always fixed. For example, it is conceivable that the width of the first display region 110 is always fixed and the hospitalization period is long. In this case, in order to grasp the hospitalization period medical information 112 as much as possible, an aspect will be adopted in which a width of each cell 114 of the first display region 110 is reduced or a part of the hospitalization period medical information 112 is not displayed, and then displayed in response to an operation, such as scrolling. However, in these aspects, the width of the cell 114 may fluctuate or a scrolling operation may be needed. In the medical support system 10 according to the third embodiment, since the width of the first display region 110 fluctuates in accordance with the hospitalization period, even in a case in which the hospitalization period is long, the fluctuation in the width of each cell 114 is suppressed (that is, the cell 114 is maintained at a predetermined width), or a range of dates not to be displayed is restricted. Therefore, with the present configuration, it is easy to grasp the hospitalization period medical information 112 of the entire hospitalization period.

It should be noted that, in the third embodiment, the form example has been described in which the width of the first display region 110 is determined in accordance with the hospitalization period, but the technology of the present disclosure is not limited to this. For example, in the present modification example, an upper limit value is set in the width of the first display region 110.

The screen generation unit 26C (see FIG. 26) adjusts the width (that is, the range in the direction of the horizontal axis B) of the first display region 110 of the display screen 108 in accordance with the hospitalization period. For example, the screen generation unit 26C derives the width of the first display region 110 by using the width derivation information (not shown). The width derivation information refers to information in which the number of dates is associated with the width of the first display region 110. Examples of the width derivation information include the table in which the number of dates is used as input and the width of the first display region 110 is used as output, or the arithmetic expression in which the number of dates is used as an independent variable and the width of the first display region 110 is used as a dependent variable. Here, the screen generation unit 26C determines whether or not the calculated width of the first display region 110 is equal to or smaller than a predetermined upper limit value. In a case in which the width of the first display region 110 is equal to or smaller than the upper limit value, the screen generation unit 26C adjusts the width of the first display region 110 in accordance with the hospitalization period.

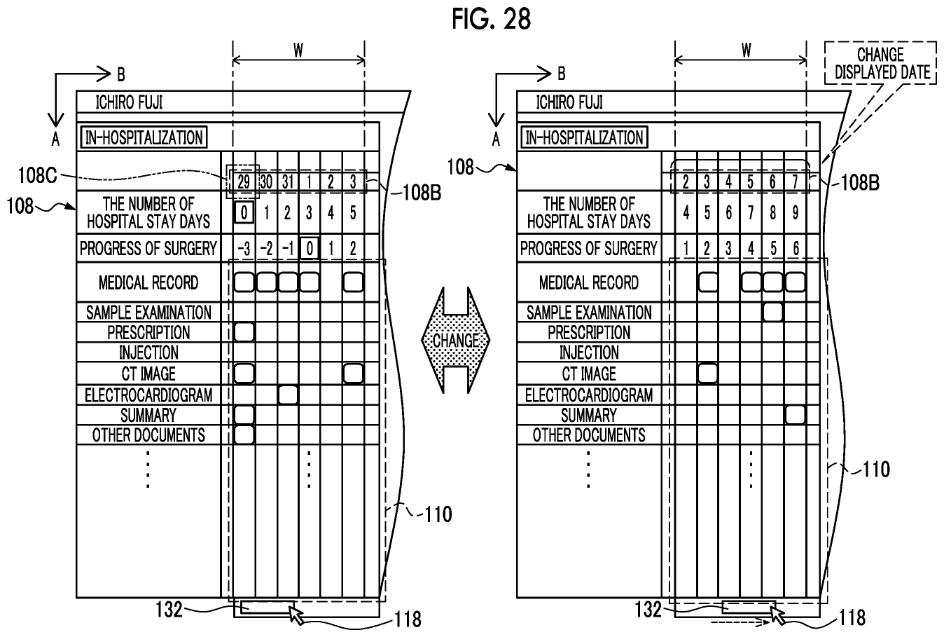

On the other hand, in a case in which the width of the first display region 110 exceeds the upper limit value (stated another way, in a case in which the hospitalization period exceeds a period corresponding to the upper limit value of the width of the first display region 110), the screen generation unit 26C uses the period corresponding to the upper limit value as the width of the first display region 110 to generate the display screen 108. In this case, the screen generation unit 26C indicates the horizontal axis B of the first display region 110 based on a designated day in the hospitalization period. Here, in an example shown in FIG. 28, an example is shown in which a basis day is the hospitalization day 108C, but this is merely an example. For example, the designated day in the hospitalization period may be a date received via the reception device 46. Moreover, the basis day may be the hospital discharge day or may be a day on which the display screen 108 is generated.

As shown in FIG. 28 as an example, in a case in which the width W of the first display region 110 exceeds the upper limit value, the width W of the first display region 110 on the display screen 108 is the period corresponding to the upper limit value. In the example shown in FIG. 28, a total of six days from the 29th to the 31st of a certain month and the 1st to the 3rd of the following month are displayed as the first display region 110. Moreover, on the display screen 108, a scroll bar 132 is displayed below the first display region 110. The scroll bar 132 corresponds to a display range of the first display region 110. The display range of the first display region 110 is changed by moving the scroll bar 132 via the pointer 118.

As an example, as shown on the right side of the front view of FIG. 28, as a result of the position of the scroll bar 132 being moved to a direction of the right side of the front view of FIG. 28 via the pointer 118, the display range of the first display region 110 is changed. In the example shown in FIG. 28, a total of six days from the 2nd to the 7th of a certain month is displayed as the first display region 110.

As described above, in a modification example according to the third embodiment, the width of the first display region 110 has the upper limit value, and in a case in which the hospitalization period exceeds the period corresponding to the upper limit value, the period which is based on the designated day in the hospitalization period and corresponds to the upper limit value is displayed in the first display region 110. Therefore, with the present configuration, it is easy to grasp the hospitalization period medical information 112 as compared with a case in which the width of the display screen 108 is always fixed. Moreover, in a case in which the upper limit value is not set and the hospitalization period is long, the width of the first display region 110 is increased, and the hospitalization period medical information 112 displayed in the first display region 110 is increased. On the other hand, in the modification example according to the third embodiment, the upper limit value of the width of the first display region 110 is set as a value designated by the user or a set value. As a result, the width of the first display region 110 can be restricted such that the user can easily visually recognize the first display region 110, and an amount of the hospitalization period medical information 112 displayed in the first display region 110 can also be restricted. Therefore, with the present configuration, as compared with a case in which the upper limit value is not set, even in a case in which the hospitalization period is long, it is easy for the user to grasp the hospitalization period medical information 112 from a bird's-eye view.

It should be noted that, in the modification example described above, the form example has been described in which the screen generation unit 26C adjusts the width W of the first display region 110 in accordance with the hospitalization period in a case in which the width W of the first display region 110 is equal to or smaller than the upper limit value, but the technology of the present disclosure is not limited to this. The width W of the first display region 110 may be, for example, a fixed value. That is, even in a case in which the hospitalization period is equal to or smaller than the upper limit value of the width W of the first display region 110, the width W of the first display region 110 may be displayed in a fixed manner. In this case, in a range other than the hospitalization period of the first display region 110, a blank region may be displayed, or the pre-hospitalization medical information 126 or the post-hospital discharge medical information 226 may be displayed.

### Fourth Embodiment

In the first to third embodiments described above, the example has been described in which one hospitalization period is displayed on the display screen 108, but the technology of the present disclosure is not limited to this. In the fourth embodiment, a case will be described in which the hospitalization period date 108B corresponding to a designated hospitalization period among the plurality of hospitalization periods is displayed on the display screen 108.

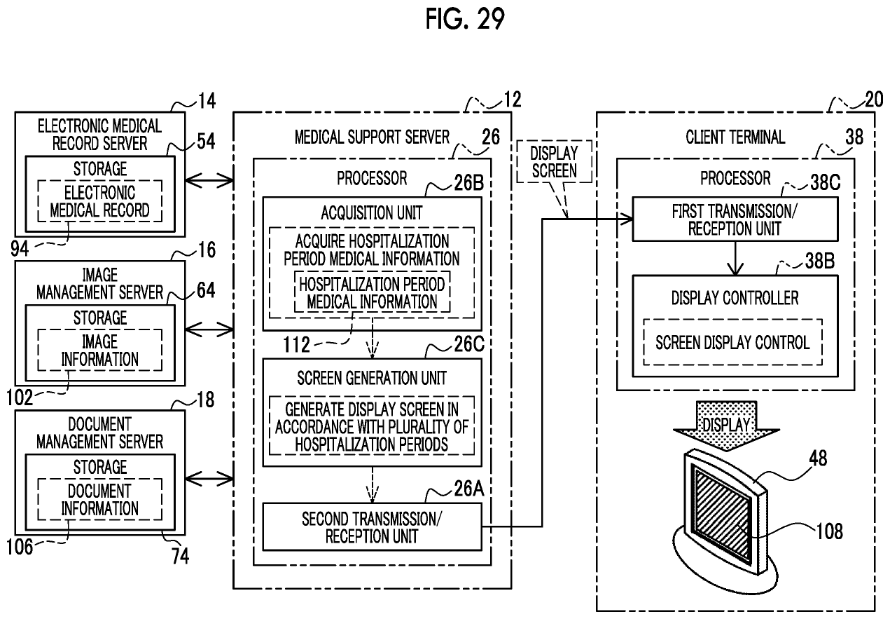

As shown in FIG. 29 as an example, the acquisition unit 26B acquires the image information 102 in accordance with a plurality of hospitalization period dates 108B from the image management server 16. Specifically, the acquisition unit 26B acquires the image information 102 in which the image date information 102B (see FIG. 7) indicating the hospitalization period date 108B is included from the image management server 16. Moreover, the acquisition unit 26B acquires the document information 106 in accordance with the hospitalization period date 108B from the document management server 18. Specifically, the acquisition unit 26B acquires the document information 106 in which the document date information 106B (see FIG. 8) indicating the hospitalization period date 108B is included from the document management server 18.

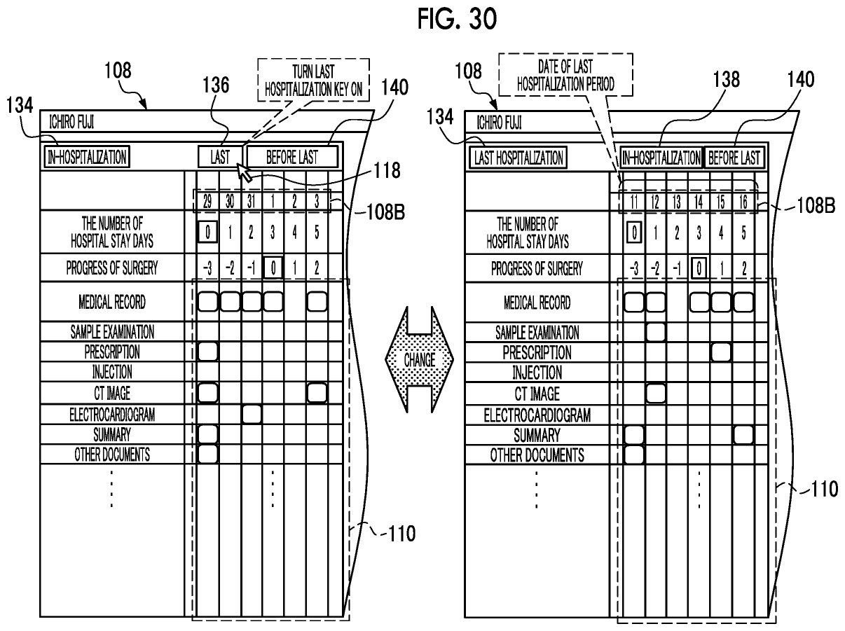

The screen generation unit 26C generates the display screen 108 (see FIG. 30) based on the electronic medical record 94, the image information 102, and the document information 106 in accordance with the plurality of hospitalization period dates 108B.

The second transmission/reception unit 26A transmits the information indicating the display screen 108 generated by the screen generation unit 26C to the client terminal 20. In the client terminal 20, the first transmission/reception unit 38C receives the information indicating the display screen 108. The display screen 108 is displayed on the display 48 under the control of the display controller 38A.

In the example shown on the left side of the front view of FIG. 30, the display screen 108 displays hospitalization period date 108B of current in-hospitalization among the plurality of hospitalization periods. In an example shown on the left side of the front view of FIG. 30, a total of six days from the 29th to the 31st of a certain month and the 1st to the 3rd of the following month are indicated on the horizontal axis B of the first display region 110. Moreover, a hospitalization period selection region 134 is displayed in the upper left corner of the display screen 108, and “in-hospitalization” is displayed in the hospitalization period selection region 134. Further, in the example shown in FIG. 30, a last hospitalization key 136 is displayed on the display screen 108. In a case in which the last hospitalization key 136 is turned on via the pointer 118, the first display region 110 is changed from the currently displayed hospitalization period date 108B of in-hospitalization to the hospitalization period date 108B corresponding to the last hospitalization period.

In the example shown on the right side of FIG. 30, the display screen 108 displays the last hospitalization period date 108B among the plurality of hospitalization periods. A total of six days from the 11th to the 16th of a certain month is shown on the horizontal axis B of the first display region 110. The hospitalization period selection region 134 is displayed in the upper left corner of the display screen 108, and “last hospitalization” is displayed in the hospitalization period selection region 134.

Moreover, in the example shown on the right side of FIG. 30, an in-hospitalization key 138 is displayed on the display screen 108. In a case in which the in-hospitalization key 138 is turned on via the pointer 118, the date is changed from the currently displayed hospitalization period date 108B to the hospitalization period date 108B of in-hospitalization.

In the example shown in FIG. 30, a hospitalization-before-last key 140 is displayed on the display screen 108. In a case in which the hospitalization-before-last key 140 is turned on via the pointer 118, the date is changed from the currently displayed hospitalization period date 108B to the hospitalization period date 108B corresponding to the hospitalization period before the last.

As described above, in the medical support system 10 according to the fourth embodiment, the acquisition unit 26B acquires the hospitalization period date 108B corresponding to the designated hospitalization period among the plurality of hospitalization periods. Therefore, with the present configuration, it is easy to grasp the hospitalization period medical information 112 as compared with a case in which the hospitalization period medical information 112 is displayed only for a predetermined hospitalization period among the plurality of hospitalization periods. Moreover, by performing the display indicating there are the plurality of hospitalization periods, such as “last” and “before the last”, it is possible to indicate whether or not a target patient has a plurality of hospitalization periods and it is possible to confirm history information of the hospitalization period of the patient as compared with a case in which there are no plurality of hospitalization periods. Further, the user can designate the specific hospitalization period by selecting the display indicating any of the plurality of hospitalization periods, such as “last” and “before the last”. Therefore, with the present configuration, it is easy for the user to designate the specific hospitalization period as compared with a case in which the plurality of displayed hospitalization periods cannot be selected.

It should be noted that, in the fourth embodiment, the form example has been described in which the hospitalization period date 108B corresponding to the hospitalization period of in-hospitalization, the last hospitalization period, and the hospitalization period before the last is switched, but the technology of the present disclosure is not limited to this. For example, the hospitalization period of the target to be switched may be a hospitalization period prior to the hospitalization period before the last, or any of the plurality of hospitalization periods may be designated via the reception device 46.

Moreover, in the fourth embodiment, the form example has been described in which the plurality of hospitalization periods are switched by turning on the last hospitalization key 136, the in-hospitalization key 138, and the hospitalization-before-last key 140 via the pointer 118, but the technology of the present disclosure is not limited to this. For example, an aspect may be adopted in which a pull-down menu indicating a list of “last hospitalization” and “hospitalization before the last” is displayed in the hospitalization period selection region 134, and the plurality of hospitalization periods are switched by designating the hospitalization period from the pull-down menu.

### Fifth Embodiment

In the first to fourth embodiments, the case has been described in which the icon 113 is designated and then the corresponding medical information 120 is displayed in the medical information display region 116, but the technology of the present disclosure is not limited to this. In the fifth embodiment, a case will be described in which the icon 113 is switched in accordance with a state of the medical information 120 displayed in the medical information display region 116.

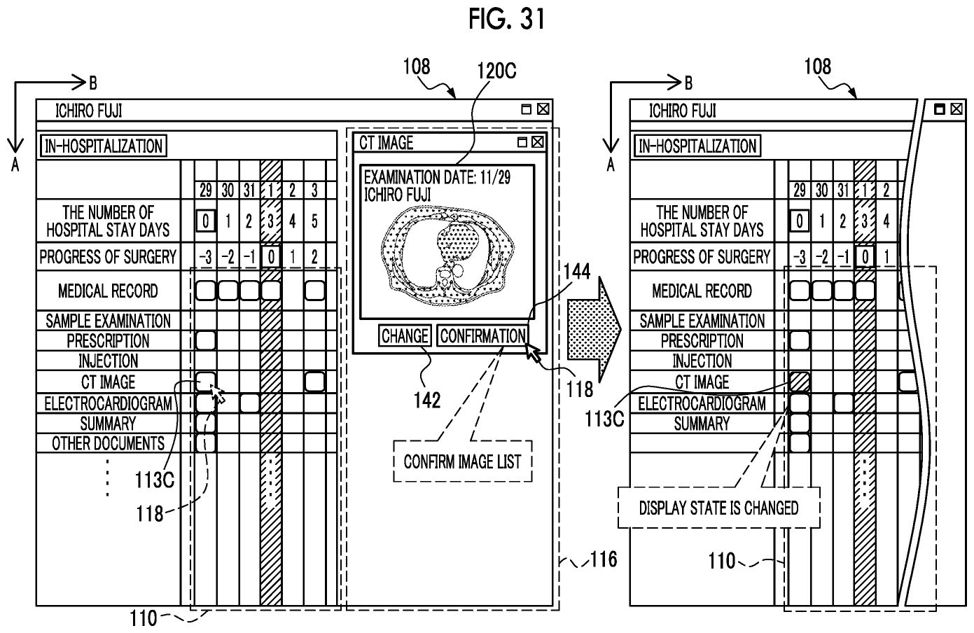

As shown in FIG. 31 as an example, in a case in which the icon 113 is designated via the pointer 118 in the first display region 110 of the display screen 108, a content indicated by the medical information 120 corresponding to the icon 113 is displayed in the medical information display region 116. In an example shown in FIG. 31, the CT image 120C indicated by the medical information 120 corresponding to the icon 113C is displayed in the medical information display region 116. In the medical information display region 116, a change key 142 for changing the state of the medical information 120 and a confirmation key 144 for confirming the state of the medical information 120 are displayed. In the example shown in FIG. 31, the change key 142 and the confirmation key 144 are displayed below the CT image 120C.

In a case in which the change key 142 is turned on via the pointer 118, the content of the medical information 120 can be changed (that is, edited). Moreover, in a case in which the confirmation key 144 is turned on via the pointer 118, it is fixed that the medical worker has confirmed the medical information 120. In the example shown in FIG. 31, the confirmation key 144 of the CT image 120C is turned on via the pointer 118. As a result, it is fixed that the medical worker has confirmed the CT image 120C.

In a case in which the confirmation key 144 is turned on, as shown on the right side of the front view of FIG. 31 as an example, the icon 113C corresponding to the CT image 120C is switched in the first display region 110. In the example shown in FIG. 31, the icon 113C is switched to a pattern different from that before the confirmation as a result of the confirmation key 144 being turned on.

As described above, in the medical support system 10 according to the fifth embodiment, the icon 113 is switched in accordance with the state of the medical information 120. Therefore, with the present configuration, it is easy to grasp the hospitalization period medical information 112 as compared with a case in which the icon 113 is always displayed in a fixed manner.

It should be noted that, in the fifth embodiment, the form example has been described in which the icon 113 is switched in accordance with the result of the state of the medical information 120 being changed or confirmed, but the technology of the present disclosure is not limited to this. The icon 113 may be switched in accordance with a result of a state of information (for example, the date information) included in the medical information 120 being changed or confirmed instead of the state of the medical information 120 itself or together with the state of the medical information 120 itself. That is, the icon 113 is switched in accordance with the information related to the medical information 120. Here, the information related to the medical information 120 includes the medical information 120 itself and accessory information of the medical information 120. For example, an aspect may be adopted in which the icon 113 is displayed in white in a case in which the date indicated by the date information included in the medical information 120 is not input and the icon 113 is colored in a case in which the date is input. That is, an aspect may be adopted in which a display aspect of the icon 113 is changed in an input state of the accessory information of the medical information 120.

As described above, in the medical support system 10 according to a modification example of the fifth embodiment, the icon 113 is switched in accordance with the state of information (for example, the date information) included in the medical information 120 instead of the state of the medical information 120 itself or together with the state of the medical information 120 itself. Therefore, with the present configuration, it is easy to grasp the hospitalization period medical information 112 as compared with a case in which the icon 113 is always displayed in a fixed manner.

Moreover, in the fifth embodiment, the form example has been described in which it is fixed that the medical information 120 has been confirmed by turning the confirmation key 144 on, but the technology of the present disclosure is not limited to this. For example, an aspect may be adopted in which it is fixed that the medical information 120 has been confirmed by closing the window in which the content indicated by the medical information 120 is displayed.

Moreover, in the fifth embodiment described above, the form example has been described in which the pattern of the icon 113 is switched after the state of the medical information 120 is changed or confirmed, but the technology of the present disclosure is not limited to this, and it is sufficient to adopt an aspect in which the user can recognize that the state of the medical information 120 is changed. For example, the color, pattern, and/or shape of the icon 113 may be changed, or a character, such as “done”, may be displayed in the icon 113.

Moreover, in the fifth embodiment described above, the form example has been described in which the icon 113 is switched after the state of the medical information 120 is changed or confirmed by the medical worker himself/herself, but the technology of the present disclosure is not limited to this, and the icon 113 may be switched in accordance with a result of confirmation by another medical worker (for example, a medical worker belonging to another medical care department or section).

### Sixth Embodiment

In the first to fifth embodiments described above, the case has been described in which the icon 113 indicates the presence of the corresponding medical information 120, but the technology of the present disclosure is not limited to this. In the sixth embodiment, a case in which the icon 113 indicates the number of medical information 120 and a case in which the icon 113 indicates the presence of information accompanying the medical information 120 will be described.

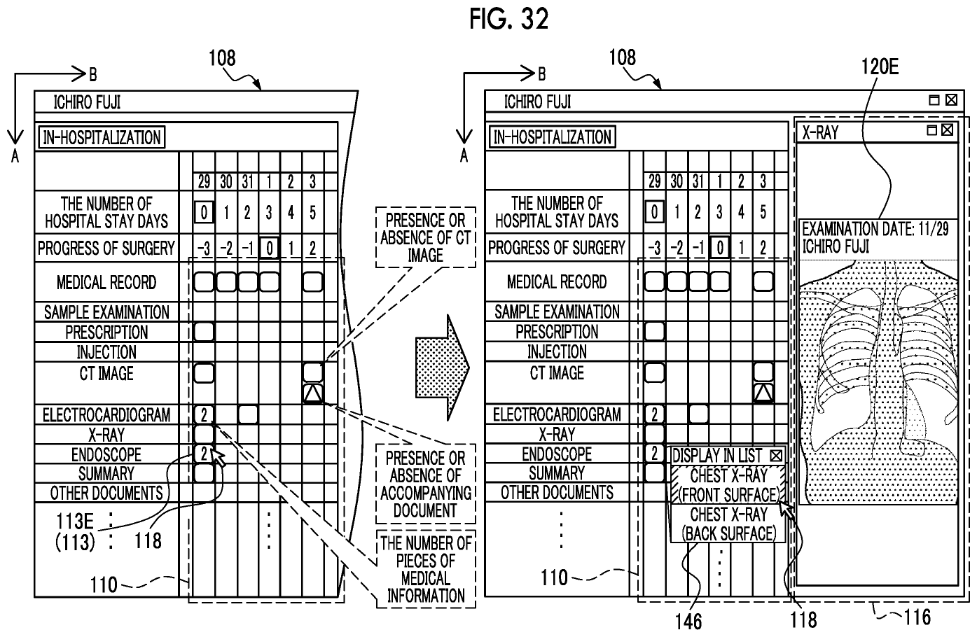

As shown in FIG. 32 as an example, the icon 113 is displayed in the first display region 110 of the display screen 108. In an example shown on the left side of FIG. 32, the number of medical information 120 corresponding to the icon 113 is displayed inside the quadrangular icon 113. In the example shown in FIG. 32, since two X-ray images 120E are associated with the icon 113E as the medical information 120, a number “2” is displayed inside the icon 113.

In a case in which the icon 113 is designated via the pointer 118, a list of the medical information 120 corresponding to the icon 113 is displayed in a pop-up display region 146, as shown on the right side of FIG. 32 as an example. In the example shown in FIG. 32, text information “chest X-ray (front surface)” and text information “chest X-ray (back surface)” are displayed in a list as the medical information 120 corresponding to the icon 113. In a case in which the medical information 120 displayed in the pop-up display region 146 is designated via the pointer 118, the designated medical information 120 is displayed in the medical information display region 116. In the example shown in FIG. 32, the X-ray image 120E corresponding to the designated “chest X-ray (front surface)” is displayed in the medical information display region 116.

As described above, in the medical support system 10 according to the sixth embodiment, the number of medical information 120 is displayed on the icon 113. Therefore, with the present configuration, it is easy to grasp the hospitalization period medical information 112 as compared with a case in which the icon 113 is always displayed in a fixed manner. Moreover, in a case in which there are a plurality of pieces of the hospitalization period medical information 112 in one cell 114 of the first display region 110, in an attempt to display the icon 113 indicating each hospitalization period medical information 112, the size of the icon 113 or the size of the cell 114 fluctuates greatly. On the other hand, by displaying numerical information together with the display of the icon 113 as one, it is possible to suppress the fluctuation in the size of the icon 113 or the size of the cell 114, and it is easy to grasp the hospitalization period medical information 112 from a bird's-eye view.

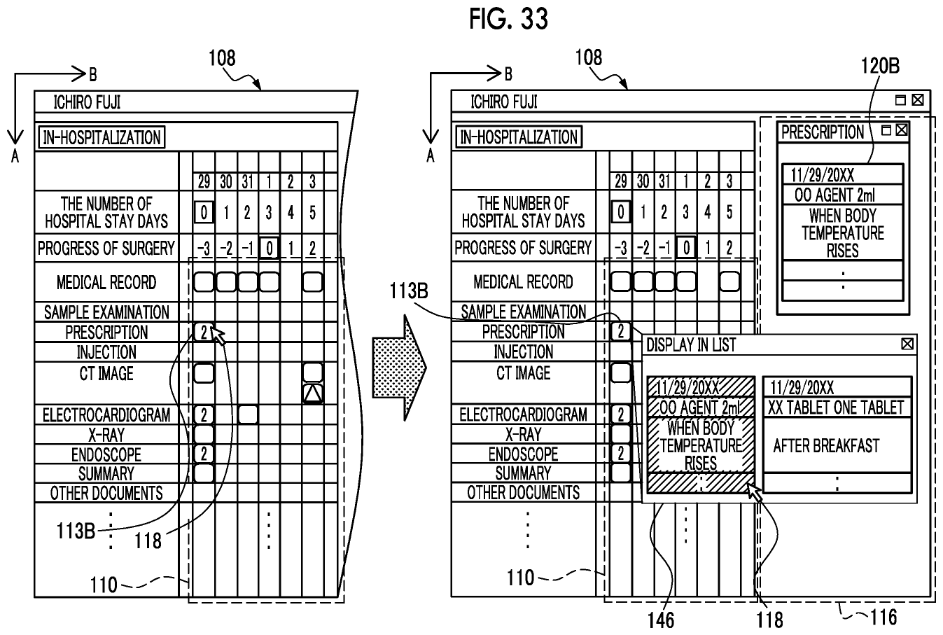

It should be noted that, in the sixth embodiment, the form example has been described in which the medical information 120 corresponding to the icon 113 is displayed in a list in the pop-up display region 146, but the technology of the present disclosure is not limited to this. As shown in FIG. 33 as an example, the medical information 120 itself may be displayed in the pop-up display region 146. In an example shown in FIG. 33, two images indicating the prescription 120B are displayed in the pop-up display region 146 as the medical information 120 corresponding to the icon 113B. In a case in which the image indicating the prescription 120B displayed in the pop-up display region 146 is designated via the pointer 118, the designated prescription 120B is displayed in the medical information display region 116.

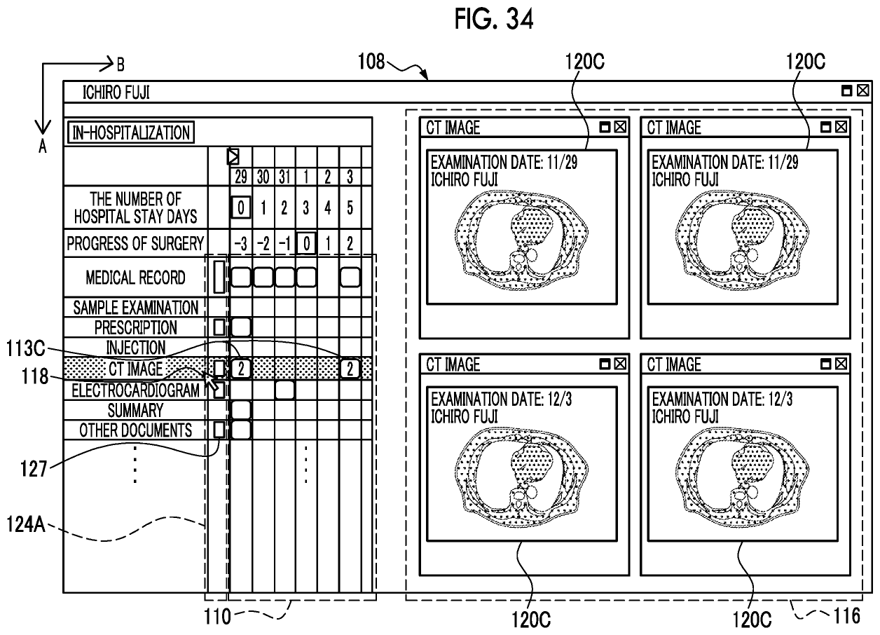

Moreover, in the sixth embodiment, the form example has been described in which the icon 113 associated with a plurality of pieces of medical information 120 is designated, but the technology of the present disclosure is not limited to this. As shown in FIG. 34 as an example, the entire row including the icon 113 associated with the plurality of pieces of medical information 120 may be designated. In this case, all of the plurality of pieces of medical information 120 corresponding to the icon 113 included in the designated row is displayed. In an example shown in FIG. 34, the entire row defined by the item of the CT image is designated. In this case, in the medical information display region 116, all of the plurality of CT images 120C indicated by the medical information 120 corresponding to the icon 113C are displayed.

It should be noted that the example has been described in which all the contents indicated by the medical information 120 corresponding to the icon 113 are displayed in a case in which the entire row is designated, but this is merely an example. For example, a latest CT image 120C among the plurality of CT images 120C may be displayed in the medical information display region 116.

Moreover, as shown in FIG. 34 as an example, in a case in which the entire row is designated in a state in which the pre-hospitalization display region 124A is displayed in a single column, the medical information 120 corresponding to the icon 127 displayed in the pre-hospitalization display region 124A does not have to be displayed in the medical information display region 116.

It should be noted that, in each of the embodiments described above, the form example has been described in which the content indicated by the medical information 120 corresponding to the icon 113 is displayed in the medical information display region 116 by designating the icon 113, but the technology of the present disclosure is not limited to this. For example, the content indicated by the medical information 120 corresponding to the cell 114 may be displayed in the medical information display region 116 by designating the cell 114.

Moreover, in each of the embodiments described above, the form example has been described in which the first display region 110 and/or the second display region 124 is displayed on the display screen 108, but the technology of the present disclosure is not limited to this. For example, the first display region 110 and/or the second display region 124 may be displayed on a screen which is different from the display screen 108.

Moreover, in each of the embodiments described above, the form example has been described in which the display screen 108 includes both the first display region 110 and the second display region 124, but the technology of the present disclosure is not limited to this. For example, on the display screen 108, the second display region 124 does not have to be displayed, and only the first display region 110 may be displayed.

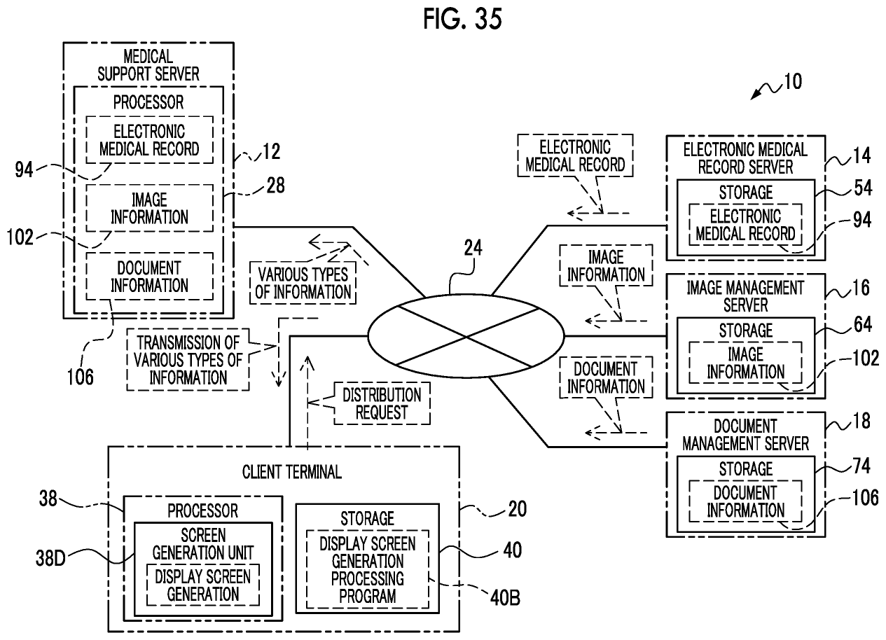

Moreover, in each of the embodiments described above, the form example has been described in which the display screen 108 is generated in the medical support server 12, but this is merely an example, and the display screen 108 may be generated in the client terminal 20, as shown in FIG. 35 as an example. In this case, for example, the client terminal 20 need only perform the same processing as the display screen generation processing and the screen display processing. That is, in the client terminal 20, a display screen generation processing program 40B is stored in the storage 40. The processor 38 reads out the display screen generation processing program 40B from the storage 40 and executes the read out display screen generation processing program 40B on the RAM 42 to perform the display screen generation processing. The display screen generation processing is realized by operating the processor 38 as a screen generation unit 38D. In this case, the client terminal 20 is an example of a “medical support device” according to the technology of the present disclosure, and the processor 38 is an example of a “processor” according to the technology of the present disclosure. Moreover, the display screen generation processing program 40B is an example of a “program” according to the technology of the present disclosure.

Moreover, at least one of the medical support server 12 or the client terminal 20 may have at least one function of the electronic medical record server 14, the image management server 16, or the document management server 18.

Moreover, in the embodiment described above, the form example has been described in which the medical support server 12 acquires the electronic medical record 94 from the electronic medical record server 14 to specify the hospitalization period date 108B, but the technology of the present disclosure is not limited to this. For example, an aspect may be adopted in which the hospitalization period information 96 corresponding to patient information is held by the electronic medical record server 14, and the patient specification information 90 is transmitted from the medical support server 12 to the electronic medical record server 14, so that the medical support server 12 acquires the hospitalization period information 96 from the electronic medical record server 14.

Moreover, in the embodiment described above, the medical support server 12 acquires the electronic medical record 94 by transmitting the patient specification information 90 to the electronic medical record server 14 by the medical support server 12 at a stage in which the medical support server 12 receives a start notification including the patient specification information 90. Moreover, the form example has been described in which the hospitalization period information 96 is acquired from the electronic medical record 94, the medical support server 12 transmits the patient specification information 90 and the hospitalization period information 96 to each of the image management server 16 and the document management server 18, so that the medical support server 12 receives the image information 102 and the document information 106, but the technology of the present disclosure is not limited to this.

For example, the medical support system 10 may include a management server (not shown) that acquires the hospitalization period information 96, the electronic medical record 94, the image information 102, and the document information 106 corresponding to a plurality of pieces of patient information from the electronic medical record server 14, the image management server 16, and the document management server 18 and manages the acquired pieces of information, the medical support server 12 or the client terminal 20 may transmit the patient specification information 90 to the management server to acquire the corresponding hospitalization period information 96, electronic medical record 94, image information 102, and document information 106. In this case, a timing at which the management server acquires any one of the hospitalization period information 96, the electronic medical record 94, the image information 102, or the document information 106 from the electronic medical record server 14, the image management server 16, and the document management server 18 may be different from a timing at which the medical support server 12 or the client terminal 20 transmits the patient specification information 90. For example, update information may be transmitted to the management server at a timing at which the server of each of the electronic medical record server 14, the image management server 16, and the document management server 18 updates the data, or the management server may periodically request each server to update the information.

Moreover, in the embodiment described above, the case has been described in which one electronic medical record server 14, one image management server 16, and one document management server 18 are provided as the server that manages the medical information 120, but the technology of the present disclosure is not limited to this, and a plurality of electronic medical record servers 14, a plurality of image management servers 16, and a plurality of document management servers 18 may be provided. Moreover, another server that manages the medical information 120 may be present instead of or together with the electronic medical record server 14, the image management server 16, and the document management server 18. Moreover, the server that manages the medical information 120 may be one server having functions equivalent to the electronic medical record server 14, the image management server 16, and the document management server 18, instead of the electronic medical record server 14, the image management server 16, and the document management server 18.

Moreover, in the embodiment described above, the form example has been shown in which the screen display processing program 40A is stored in the storage 40, but the technology of the present disclosure is not limited to this. For example, the screen display processing program 40A may be stored in a storage medium, such as an SSD or a USB memory. The storage medium is a portable computer-readable non-transitory storage medium. The screen display processing program 40A which is stored in the storage medium is installed in the client terminal 20. The processor 38 executes the screen display processing in accordance with the screen display processing program 40A.

Moreover, the screen display processing program 40A may be stored in a storage device of another computer or server connected to the client terminal 20 via the network 24 (see FIG. 1), and the screen display processing program 40A may be downloaded in response to a request of the client terminal 20 and installed in the client terminal 20.

It is not necessary to store the entire screen display processing program 40A in the storage device of the other computer or server connected to the client terminal 20 or the storage 40, and a part of the screen display processing program 40A may be stored. It should be noted that the storage medium, the storage device of the other computer or server connected to the client terminal 20, and another external storage are positioned as a memory used by being directly or indirectly connected to the processor 38.

Moreover, in the embodiment described above, the processor 38, the storage 40, the RAM 42, and the communication I/F 44 of the client terminal 20 are described as the computer, but the technology of the present disclosure is not limited to this, and a device including an application specific integrated circuit (ASIC), a field-programmable gate array (FPGA), and/or a programmable logic device (PLD) may be applied instead of the computer. Moreover, a hardware configuration and a software configuration may be used in combination, instead of the computer.

As the hardware resource for executing the screen display processing described in the embodiment above, the following various processors can be used. Examples of the processor include a CPU, which is a general-purpose processor that functions as the hardware resource for executing the screen display processing by executing the software, that is, the program. Moreover, examples of the processor include a dedicated electric circuit which is a processor having a circuit configuration designed to be dedicated to executing specific processing, such as an FPGA, a PLD, or an ASIC. A memory is also built in or connected to any processor, and any processor executes the screen display processing by using the memory.

The hardware resource for executing the screen display processing may be composed of one of those various processors or may be composed of a combination of two or more processors of the same type or different types (for example, a combination of a plurality of FPGAs or a combination of a CPU and an FPGA). Moreover, the hardware resource for executing the screen display processing may be one processor.

As one example of the configuration with one processor, first, there is a form in which one processor is composed of a combination of one or more CPUs and software, and this processor functions as the hardware resource for executing the screen display processing. Second, as represented by system-on-a-chip (SoC), there is a form in which a processor that realizes the functions of the entire system including a plurality of hardware resources for executing the screen display processing with a single integrated circuit (IC) chip is used. As described above, the screen display processing is realized by using one or more of the various processors described above as the hardware resources.

As a hardware structure of these various processors, more specifically, it is possible to use an electric circuit in which circuit elements, such as semiconductor elements, are combined. Moreover, the screen display processing is merely an example. Therefore, it is needless to say that unnecessary steps may be deleted, new steps may be added, or a processing order may be changed within a range that does not deviate from the gist.

Moreover, in the embodiment described above, the form example has been described in which the display screen generation processing program 28A is stored in the storage 28, but the technology of the present disclosure is not limited to this. For example, the display screen generation processing program 28A may be stored in a storage medium, such as an SSD or a USB memory. The storage medium is a portable computer-readable non-transitory storage medium. The display screen generation processing program 28A which is stored in the storage medium is installed in the medical support server 12. The processor 26 executes the display screen generation processing in accordance with the display screen generation processing program 28A.

Moreover, the display screen generation processing program 28A may be stored in a storage device of another computer or server connected to the medical support server 12 via the network 24 (see FIG. 1), and the display screen generation processing program 28A may be downloaded in response to a request of the medical support server 12 and installed in the medical support server 12.

It is not necessary to store the entire display screen generation processing program 28A in the storage device of the other computer or server connected to the medical support server 12 or the storage 28, and a part of the display screen generation processing program 28A may be stored. It should be noted that the storage medium, the storage device of the other computer or server connected to the medical support server 12, and another external storage are positioned as a memory used by being directly or indirectly connected to the processor 26.

Moreover, in the embodiment described above, the medical support server 12 is described as the computer, but the technology of the present disclosure is not limited to this, and a device including an application specific integrated circuit (ASIC), a field-programmable gate array (FPGA), and/or a programmable logic device (PLD) may be applied instead of the computer. Moreover, a hardware configuration and a software configuration may be used in combination, instead of the computer.

As the hardware resource for executing the display screen generation processing described in the embodiment above, the following various processors can be used. Examples of the processor include a CPU, which is a general-purpose processor that functions as the hardware resource for executing the display screen generation processing by executing the software, that is, the program. Moreover, examples of the processor include a dedicated electric circuit which is a processor having a circuit configuration designed to be dedicated to executing specific processing, such as an FPGA, a PLD, or an ASIC. A memory is also built in or connected to any processor, and any processor executes the display screen generation processing by using the memory.

The hardware resource for executing the display screen generation processing may be composed of one of those various processors or may be composed of a combination of two or more processors of the same type or different types (for example, a combination of a plurality of FPGAs or a combination of a CPU and an FPGA). Moreover, the hardware resource for executing the display screen generation processing may be one processor.

As one example of the configuration with one processor, first, there is a form in which one processor is composed of a combination of one or more CPUs and software, and this processor functions as the hardware resource for executing the display screen generation processing. Second, as represented by system-on-a-chip (SoC), there is a form in which a processor that realizes the functions of the entire system including a plurality of hardware resources for executing the display screen generation processing with a single integrated circuit (IC) chip is used. As described above, the display screen generation processing is realized by using one or more of the various processors described above as the hardware resources.

As a hardware structure of these various processors, more specifically, it is possible to use an electric circuit in which circuit elements, such as semiconductor elements, are combined. Moreover, the display screen generation processing is merely an example. Therefore, it is needless to say that unnecessary steps may be deleted, new steps may be added, or a processing order may be changed within a range that does not deviate from the gist.

The described contents and the shown contents above are the detailed description of the parts according to the technology of the present disclosure, and are merely examples of the technology of the present disclosure. For example, the description of the configuration, the function, the action, and the effect above are the description of examples of the configuration, the function, the action, and the effect of the parts according to the technology of the present disclosure. Accordingly, it is needless to say that unnecessary parts may be deleted, new elements may be added, or replacements may be made with respect to the described contents and the shown contents above within a range that does not deviate from the gist of the technology of the present disclosure. Moreover, in order to avoid complications and facilitate understanding of the parts according to the technology of the present disclosure, the description of common technical knowledge or the like, which does not particularly require the description for enabling the implementation of the technology of the present disclosure, is omitted in the described contents and the shown contents above.

In the present specification, “A and/or B” is synonymous with “at least one of A or B”. That is, “A and/or B” means that it may be only A, only B, or a combination of A and B. Moreover, in the present specification, in a case in which three or more matters are associated and expressed by “and/or”, the same concept as “A and/or B” is applied.

All documents, patent applications, and technical standards described in the present specification are incorporated into the present specification by reference to the same extent as in a case in which the individual documents, patent applications, and technical standards are specifically and individually stated to be described by reference.

What is claimed is:

1. A medical support device comprising:
at least one processor,
that is configured to:
acquire hospitalization period medical information including a hospitalization period date, which is a date corresponding to a hospitalization period, and a plurality of first items associated with the hospitalization period date, and
generate a display screen including a first display region defined by a first axis indicating the hospitalization period date and a second axis indicating the plurality of first items and intersecting with the first axis, in which the hospitalization period medical information is disposed in a matrix in the first display region.

2. The medical support device according to claim 1,
wherein, in the display screen, the number of days based on a hospitalization day is displayed on at least one date included in the hospitalization period dates.

3. The medical support device according to claim 1,
wherein, in the display screen, a surgery day is displayed to be distinguishable from a date other than the surgery day.

4. The medical support device according to claim 1,
wherein, in the display screen, the number of days based on a surgery day is displayed on at least one date included in the hospitalization period.

5. The medical support device according to claim 1,
wherein the at least one processor is configured to:
acquire non-hospitalization period medical information including a non-hospitalization period date, which is a date corresponding to an outside of the hospitalization period, and a plurality of second items associated with the non-hospitalization period date, and
generate a second display region defined by a third axis indicating the non-hospitalization period date and a fourth axis indicating the plurality of second items and intersecting with the third axis, and
in the second display region, the non-hospitalization period medical information is disposed in a matrix.

6. The medical support device according to claim 5,
wherein the non-hospitalization period date is determined based on any of a start day or an end day of the hospitalization period.

7. The medical support device according to claim 5,
wherein, in the second display region, the third axis indicates the date for a first number which is a number smaller than the number of the dates included in the non-hospitalization period dates.

8. The medical support device according to claim 7,
wherein, in the second display region, the number of the dates indicated by the third axis is switchable between the first number and a second number larger than the first number.

9. The medical support device according to claim 5,
wherein, in the second display region, the third axis indicates the date on which the non-hospitalization period medical information is disposed among the non-hospitalization period dates.

10. The medical support device according to claim 5,
wherein the second display region includes a pre-hospitalization display region and a post-hospital discharge display region, and
in a case in which an end day of the hospitalization period is present, the post-hospital discharge display region is displayed.

11. The medical support device according to claim 1,
wherein a range of the first display region in a direction of the first axis is determined in accordance with the hospitalization period.

12. The medical support device according to claim 1,
wherein a range of the first display region in a direction of the first axis has an upper limit value, and
in a case in which the hospitalization period exceeds a period corresponding to the upper limit value, a period, which is based on a designated day in the hospitalization period and corresponds to the upper limit value, is displayed in the first display region.

13. The medical support device according to claim 1,
wherein the at least one processor is configured to acquire the hospitalization period medical information including the hospitalization period date corresponding to a designated hospitalization period among a plurality of the hospitalization periods, and the plurality of first items corresponding to the hospitalization period date.

14. The medical support device according to claim 1,
wherein, in the first display region, an icon indicating that medical information is present is displayed in a cell specified by the date and the first item, and
the at least one processor is configured to generate a medical information display region in which the medical information corresponding to a designated icon and/or a designated cell is displayed.

15. The medical support device according to claim 14,
wherein the icon is switched in accordance with a state of information related to the medical information.

16. The medical support device according to claim 15,
wherein the state includes a state of the medical information itself and/or a state of accessory information of the medical information.

17. The medical support device according to claim 14,
wherein the number of pieces of the medical information is displayed on the icon.

18. The medical support device according to claim 14,
wherein, in the medical information display region, the medical information is displayed in any of a designation order of the icon and/or the cell, a date order, or an arrangement order of the first items in a direction of the second axis.

19. The medical support device according to claim 1,
wherein the at least one processor is configured to output information for displaying the display screen on a display device.

20. A display device that displays the display screen generated in the medical support device according to claim 1.

21. A medical support method comprising:
acquiring hospitalization period medical information including a hospitalization period date, which is a date corresponding to a hospitalization period, and a plurality of first items associated with the hospitalization period date; and
generating a display screen including a first display region defined by a first axis indicating the hospitalization period date and a second axis indicating the plurality of first items and intersecting with the first axis, in which the hospitalization period medical information is disposed in a matrix in the first display region.

22. A non-transitory storage medium storing a program causing a computer to execute a process, the process comprising:
acquiring hospitalization period medical information including a hospitalization period date, which is a date corresponding to a hospitalization period, and a plurality of first items associated with the hospitalization period date; and
generating a display screen including a first display region defined by a first axis indicating the hospitalization period date and a second axis indicating the plurality of first items and intersecting with the first axis, in which the hospitalization period medical information is disposed in a matrix in the first display region.
